(19) United States
(12) Patent Application Publication
(10) Pub. No.: US 2016/0355465 A1
(43) Pub. Date: Dec. 8, 2016

(54) NOVEL MODIFIED CURCUMINS AND THEIR USES

(71) Applicants: Lorne M. GOLUB, Smithtown, NY (US); Hsi-ming LEE, East Setauket, NY (US); Francis JOHNSON, Setauket, NY (US)

(72) Inventors: Lorne M. GOLUB, Smithtown, NY (US); Hsi-ming LEE, East Setauket, NY (US); Francis JOHNSON, Setauket, NY (US)

(73) Assignees: THE RESEARCH FOUNDATION OF STATE UNIVERSITY OF NEW YORK, ALBANY, NY (US); CHEM-MASTER INTERNATIONAL, INC., EAST SETAUKET, NY (US)

(21) Appl. No.: 15/240,630

(22) Filed: Aug. 18, 2016

Related U.S. Application Data
(62) Division of application No. 14/352,277, filed on Apr. 16, 2014.
(60) Provisional application No. 61/548,035, filed on Oct. 17, 2011.

(51) Int. Cl. C07C 235/80

(52) U.S. Cl. CPC C07C 235/80

(57) ABSTRACT

This invention provides a compound having the structure

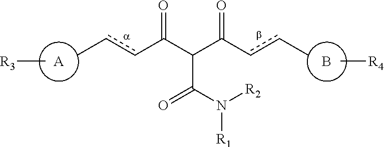

wherein α, β, A, B, and R₁-R₄ are defined herein. This invention also provides pharmaceutical compositions comprising the above compounds, a method of accelerating the healing of a wound, a method of inhibiting the activity and/or levels of a matrix metalloproteinase (MMP), a method of inhibiting the production of a cytokine in a population of cells, a method of inhibiting the production of a growth factor in a population of cells, and a method of inhibiting NFκ-B activation in a population of cells.

This application claims priority of U.S. Provisional Application No. 61/548,035, filed Oct. 17, 2011, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Curcumin (diferuloylmethane), the major component in curcuma/turmeric, is an antioxidant polyphenol from the plant Curcuma longa and is commonly used as a spice component. Curcumin has been used to treat inflammation and exerts antiproliferative and proapoptotic effects against various tumors in vitro and in vivo, and it has been found to suppress carcinogenesis of the breast and other organs (9, 10). Bachmeier and coworkers have reported downregulation of the inflammatory cytokines CXCL1 and CXCL2 in breast cancer cells via NFκB (9). Oral curcumin efficacy in vivo has been shown in models for many conditions with oxidative damage and inflammation, including many types of cancer, diabetes, atherosclerosis, arthritis, stroke, peripheral neuropathy, inflammatory bowel disease, and brain trauma (11). Curcumin, along with its tetrahydro derivative, tetrahydrocurcumin, has been shown to inhibit IL-1β in an acute brain inflammation model while curcumin was more effective than THC in attenuating plaque pathogenesis in studies of curcumin efficacy in models of neuroinflammation, which is implicated in the pathogenesis of many neurodegenerative disorders, including Alzheimer's disease (AD) (11).

Curcumin has been known to be useful in the treatment of skin disorders, including, but not limited to, wounds, psoriasis, acne, burns, eczema, as well as inflammation accompanying such disorders (20-24). Singer and co-workers have shown that curcumin reduces burn progression in rats (21) and Sidhu and co-workers have shown curcumin to be effective in enhancing wound healing in animals (22), including streptozoticin-induced diabetic rats and genetically diabetic mice (23). In addition, Phan and co-workers have shown that curcumin exhibits powerful inhibition against hydrogen peroxide damage in human keratinocytes and fibroblasts (24).

While curcumin has been shown to have multiple beneficial effects, its poor oral absorption and lack of solubility in physiological fluid has all but precluded its use as a medicinal substance. Therefore, novel chemically-modified curcumins with enhanced pharmacokinetic and pharmacodynamic properties are needed.

### SUMMARY OF THE INVENTION

This invention provides a compound having the structure

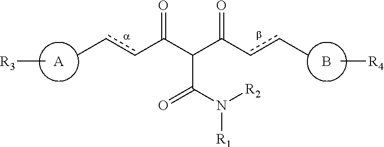

wherein
bond α and β are each, independently, present or absent;
A is an aromatic or heteroaromatic ring; B is an aromatic or heteroaromatic ring;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

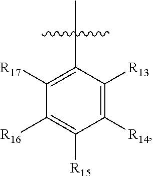

wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂O, —CSR₂₀, —NHCOR₁₈, —SOR₁₈, —POR₁₇, —C(═S) R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
   wherein R₂₁ is, H, —CF₃, —CH₃, C₁₋₅ alkyl, C₂₋₅ alkenyl, C₂₋₅ alkynyl, or —C(═O)-heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted; and
when α and β are present, A is phenyl with R₃ at the para position, B is phenyl with R₄ at the para position, R₂ is H, R₃ is —OH or —N(CH₃)₂, and R₄ is —OH or —N(CH₃)₂, then R₁ is other than unsubstituted phenyl,
or a salt thereof.

This invention provides method of accelerating the healing of a wound in a subject having a wound, comprising administering to the subject a compound having the structure

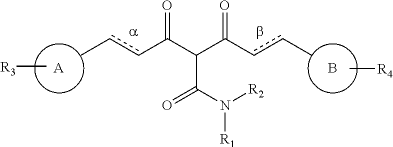

wherein
bond α and β are each, independently, present or absent;
A is an aromatic or heteroaromatic ring; B is an aromatic or heteroaromatic ring;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

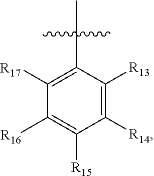

wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NR₁₈) R₁₈, —C(═N)R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
   wherein R₂₁ is, H, —CF₃, —CH₃, C₁₋₅ alkyl, C₂₋₅ alkenyl, C₂₋₅ alkynyl, or —C(═O)-heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

### BRIEF DESCRIPTION OF THE FIGURES

FIG. 1. 6 mm skin biopsy wounds in a typical normal (NDC) or diabetic rat at the time of circular biopsy (before treatment: time=0).

FIG. 2. Blood glucose levels in normal (NDC) rats and in diabetic vehicle treated controls (D), diabetic topically treated wounds with 1% and 3% Compound 4, respectively and systemically treated diabetic rats with 30 mg/kg Compound 4. Each value is the mean±S.E.M of 3 rats/group.

FIG. 3. Effect of topical and systemic administration of Compound 4. Examples of the clinical appearance of standardized skin wounds in normal (NDC) rats and in diabetic vehicle treated controls (D), topically treated diabetic wounds with 1% and 3% Compound 4, respectively and systemically treated diabetic rats with 30 mg/kg Compound 4, after 7 days of healing.

FIG. 4. Healing (% reduction in diameter) of skin wounds in normal (NDC), diabetic vehicle treated controls (D), topically treated diabetic wounds with 1% and 3% Compound 4, respectively and systemically treated diabetic rat samples treated with 30 mg/kg Compound 4. Each value is the mean±S.E.M. for 18 measurements.

FIG. 5. Diabetes increases MMP-9 (92 kDa gelatinase) but not MMP-2 (72 kDa gelatinase) in skin wounds. Effect of topical & systemic administration of Compound 4. Lane 1 shows the 92 kD and 72 kD latent/pro-forms of MMP-9 & MMP-2, respectively (standards). Lane 2 shows a normal non-diabetic control rat sample, lanes 3 and 4 show the MMP expression in diabetically induced rats. Lanes 5 and 6 reveal the reduction in MMP-9 after treatment with 1% Compound 4, while lanes 7 and 8 show the expression of MMP-2 and MMP-9 after treatment with 3% Compound 4. Finally, lanes 9 and 10 show samples extracted from rat wounds treated systemically with 30 mg/kg of Compound 4.

FIG. 6. Densitometric analysis of gelatin zymograms.

FIG. 7. The effect of topical and systemic treatment with Compound 4. Histology (H&E Staining) of skin wounds after 7 days of treatment with Compound 4 in normal & diabetic rats.

FIG. 8. Diabetes significantly suppressed re-epithelialization of standardized skin wounds. The effect of topical and systemic Compound 4. Each value represents the mean±SEM of 12 histomorphometric analysis/group.

FIG. 9. Trichrome staining for collagen. Indicates: (a) that diabetes delays wound healing in skin, and (b) that 1% Compound 4 is more effective than the other treatments in “normalizing” wound healing.

FIG. 10. Histological measurement of wound diameter (Trichrome Staining) after 7 days healing in normal and diabetic rats. Each value represents the mean±SEM of 12 measurements/group.

FIG. 11. Proposed model for wound healing mechanism using Compound 4. Diabetes is proposed to decrease inflammatory cell “competence” (e.g. decreased MO chemotactic activity) prolonging the inflammatory phase and delaying connective tissue repair. Without wishing to be bound by any scientific theory, Compound 4 is proposed to “normalize” inflammatory cell “competence”, resolve the inflammatory phase and restore collagen formation. Diabetes decreases MØ chemotactic activity, increases accumulation of MØ in exudates (note increased area-under-the-curve from MØ in diabetic rats) and increases MMPs and PICs (see, e.g. Example 7).

FIG. 12. The Effect of oral administration of Compound 4 (30 mg/kg; 21 days) on macrophage accumulation in peritoneal exudates of diabetic rats. (Each value represents the mean of 3 rats/group ±S.E.M. Note: Compound 4 did NOT affect the severity of hyperglycemia in the diabetics).

FIG. 13. Oral administration of Compound 4 in diabetic rats: effect on IL-6 production by peritoneal macrophages in cell culture with (b), and without (a), stimulation by P. gingivalis LPS (Note: Compound 4 was administered in vivo in both (a) and (b))

FIG. 14. Oral administration of Compound 4 in diabetic rats: effect on IL-1(production by peritoneal macrophages in cell culture with (b), or without (a) stimulation by P. gingivalis LPS (Note: Compound 4 was administered in vivo in both (a) and (b))

FIG. 15. Oral administration of Compound 4 in diabetic rats: effect on MMP-9 levels in conditioned media of peritoneal macrophages in cell culture (no LPS added).

FIG. 16. Oral administration of Compound 4 in diabetic rats: effect on cell migration (chemotaxis) of peritoneal macrophages in cell culture.

### DETAILED DESCRIPTION OF THE INVENTION

This invention provides a compound having the structure

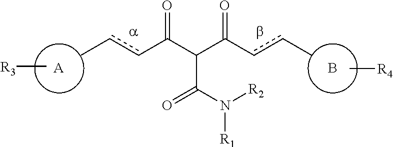

wherein
bond α and β are each, independently, present or absent;
A is an aromatic or heteroaromatic ring; B is an aromatic or heteroaromatic ring;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

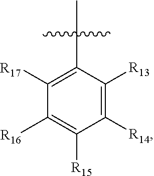

wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
   wherein R₂₁ is, H, —CF₃, —CH₃, C₁₋₅ alkyl, C₂₋₅ alkenyl, C₂₋₅ alkynyl, or —C(═O)-heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
   wherein each occurrence in the compound of alkyl, alkenyl, or alkynyl is branched or unbranched, unsubstituted or substituted; and
   when α and β are present, A is phenyl with R₃ at the para position, B is phenyl with R₄ at the para position, R₂ is H, R₃ is —OH or —N(CH₃)₂, and R₄ is —OH or —N(CH₃)₂, then R₁ is other than unsubstituted phenyl,
      or a salt thereof.

In an embodiment, the compound has the structure

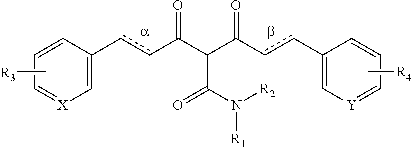

wherein
bond α and β are each, independently, present or absent;
X is —CH, —CR₃ or N; Y is —CH, —CR₄ or N;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

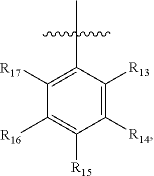

wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
   wherein R₂₁ is, H, —CF₃, —CH₃, C₁₋₅ alkyl, C₂₋₅ alkenyl, C₂₋₅ alkynyl, or —C(═O)-heterocyclyl; and
R₃ and R₄ are each independently, halogen, —NO₂, —NR₂₂R₂₃, —NHR₂₂R₂₃⁻, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted; and
when α and β are present, X is —CH, Y is —CH, R₂ is H, R₃ is —OH or —N(CH₃)₂, and R₄ is —OH or —N(CH₃)₂, then R₁ is other than unsubstituted phenyl,
   or a salt thereof.

In an embodiment, X is N, or a salt thereof.

In an embodiment, α and β are both present, or a salt thereof.

In an embodiment, the compound has the structure

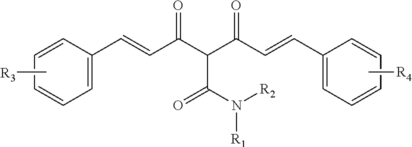

wherein
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

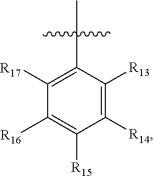

wherein R₁₃, R₁₄, R₁₅, R₁₆ and R₁₇ and are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH)R₁₈, C(═NR₁₈)R₁₈, —C(═N)R₁₈, —POR₁₈, —P(═O) (OR₁₈) (OR₁₉), —P(OR₁₈) (OR₁₉), —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H or —CH₃; and
R₃ and R₄ are each —OR₂₂,
   wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)— heterocyclyl, and
wherein each occurrence in the compound of alkyl, alkenyl, or alkynyl is branched or unbranched, unsubstituted or substituted,
   or a salt thereof.

In an embodiment, the compound has the structure

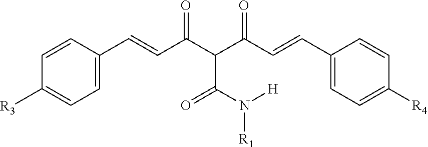

wherein
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

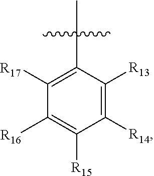

wherein R₁₃, R₁₄, R₁₅, R₁₆ and R₁₇ and are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH)R₁₈, C(═NR₁₈)R₁₈, —C(═N)R₁₈, —POR₁₈, —P(═O) (OR₁₈) (OR₁₉), —P(OR₁₈) (OR₁₉), —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each —OR₂₂,
   wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)— heterocyclyl, and
wherein each occurrence in the compound of alkyl, alkenyl, or alkynyl is branched or unbranched, unsubstituted or substituted,
   or a salt thereof.

The compound of claim 4 or 5, wherein R₂₂ is H, C₁₋₅ alkyl, C₂₋₅ alkenyl, C₂₋₅ alkynyl, aryl, heteroaryl, heterocyclyl, or —C(═O)-heterocyclyl, wherein each occurrence of alkyl, alkenyl, or alkynyl is branched or unbranched, unsubstituted or substituted, or a salt thereof.

In an embodiment, the compound has the structure

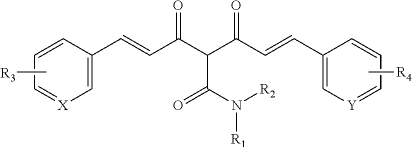

wherein
X is —CH, —CR₃ or N; Y is —CH, —CR₄ or N;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, or heterocyclyl;
R₂ is H or —CH₃; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂), or —P(OR₂₂) (OR₂₃)
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
   or a salt thereof.

In an embodiment, the compound has the structure

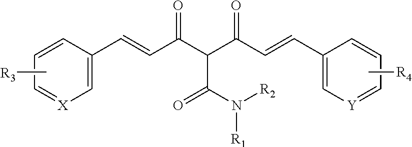

wherein
X is —CH, —CR₃ or N; Y is —CH, —CR₄ or N;
R₁ is

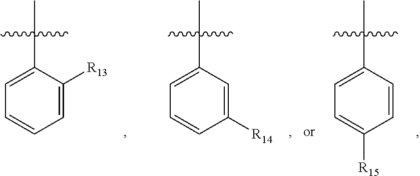

wherein R₁₃, R₁₄, or R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₈, —C(═S) R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H or —CH₃; and
R₃ and R₄ are each independently, halogen, —NO₂, —NR₂₂R₂₃, —NHR₂₂R₂₃, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
   or a salt thereof.

In an embodiment, the compound has the structure

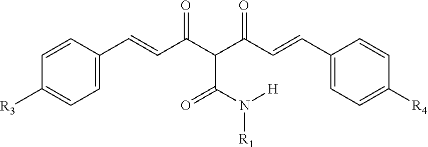

wherein
R₁ is

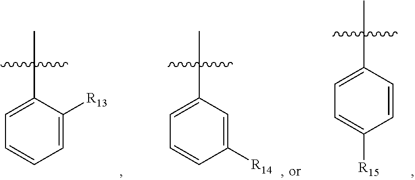

wherein R₁₃, R₁₄, or R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

In an embodiment,
R₁ is

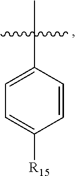

wherein R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH)R₁₈, C(═NR₁₈)R₁₈, —C(═N)R₁₈, —POR₁₈, —P(═O) (OR₁₈) (R₁₉), —P(OR₁₈) (OR₁₉), —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

In an embodiment, R₃ and R₄ are each —OR₂₂, wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and wherein each occurrence in the compound of alkyl, alkenyl, or alkynyl is branched or unbranched, unsubstituted or substituted,
or a salt thereof.

In an embodiment, R₂₂ is H.

In an embodiment, the compound has the structure

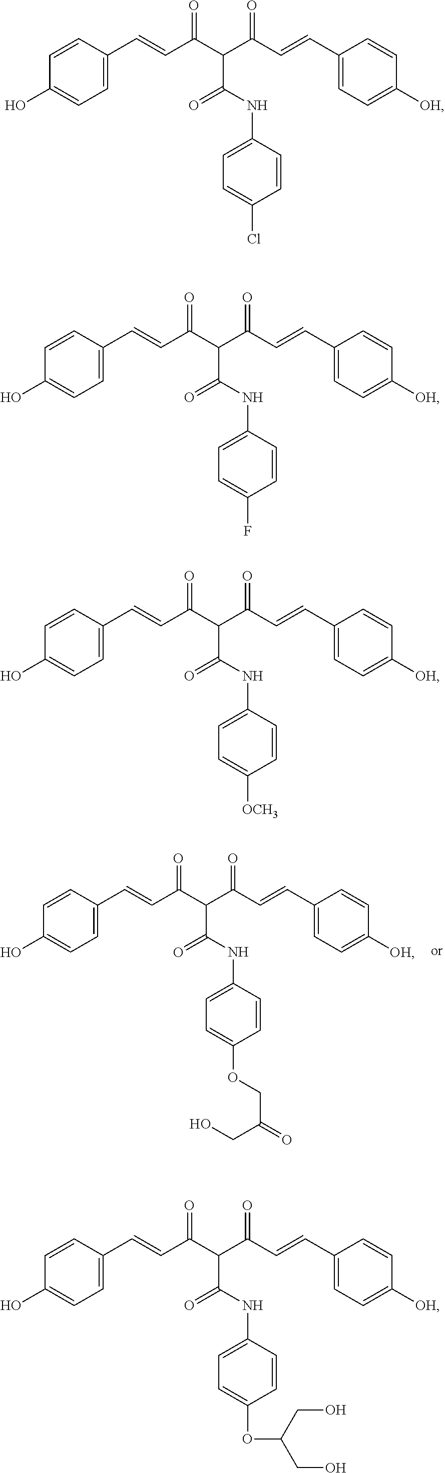

or a salt thereof.

This invention provides a pharmaceutical composition comprising a pharmaceutically acceptable carrier and one of the compounds described above.

The compounds described above are referred to herein as “Compound 4 homologues”. 10 This invention provides method of accelerating the healing of a wound in a subject having a wound, comprising administering to the subject a compound having the structure

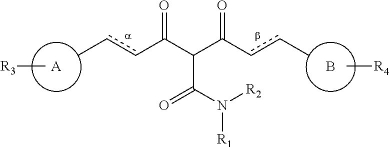

wherein
bond α and β are each, independently, present or absent;
A is an aromatic or heteroaromatic ring; B is an aromatic or heteroaromatic ring;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

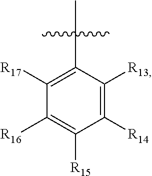

wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
   wherein R₂₁ is, H, —CF₃, —CH₃, C₁₋₅ alkyl, C₂₋₅ alkenyl, C₂₋₅ alkynyl, or —C(═O)-heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

In an embodiment, the compound administered has the structure

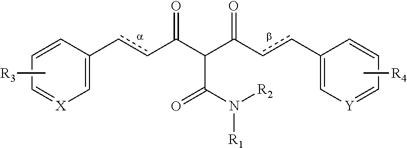

wherein
bond α and β are each, independently, present or absent;
X is —CH, —CR₃ or N; Y is —CH, —CR₄ or N;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

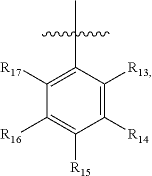

wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
   wherein R₂₁ is, H, —CF₃, —CH₃, C₁₋₅ alkyl, C₂₋₅ alkenyl, C₂₋₅ alkynyl, or —C(═O)-heterocyclyl; and R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

In an embodiment, when α and β are present, X is —CH, Y is —CH, R₂ is H, R₃ is —N(CH₃)₂ and R₄ is —N(CH₃)₂, then R₁ is other than unsubstituted phenyl.

In an embodiment, the compound administered has the structure

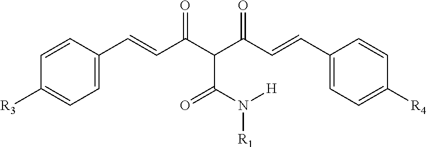

wherein
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or

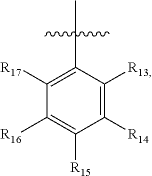

wherein R₁₃, R₁₄, R₁₅, R₁₆ and R₁₇ and are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH)R₁₈, C(═NR₁₈)R₁₈, —C(═N)R₁₈, —POR₁₈, —P(═O) (OR₁₈) (R₁₉), —P(OR₁₈) (OR₁₉), —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each —OR₂₂,
   wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)— heterocyclyl, and
wherein each occurrence in the compound of alkyl, alkenyl, or alkynyl is branched or unbranched, unsubstituted or substituted,
or a salt thereof.

In an embodiment, the compound administered has the structure

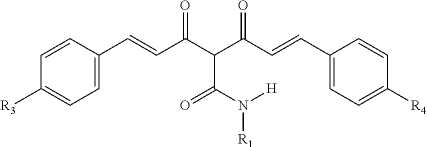

wherein
R₁ is

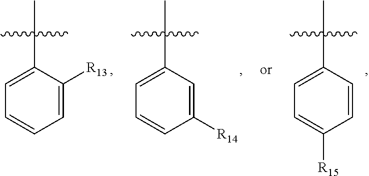

wherein R₁₃, R₁₄, or R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NR₁₈₎R₁₈, —C(═N) R₁₈, —POR₁₈, —P(═O) (OR₁₈) (OR₁₉), —P(OR₁₈) (OR₁₉) r, —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃) or —P(OR₂₂) (OR₂₃),
   wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

In an embodiment,
R₁ is

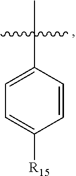

wherein R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH)R₁₈, C(═NR₁₈)R₁₈, —C(═N)R₁₈, —POR₁₈, —P(═O) (OR₁₈) (OR₁₉), —P(OR₁₈) (OR₁₉), —C(═S) R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
   wherein Z is halogen, and
   R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
   wherein R₂₂ and R₂₃ are each, H, —CF₃, —CH₃, C₁₋₅alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

In an embodiment, R₃ and R₄ are each —OR₂₂, wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)-heterocyclyl,

wherein each occurrence in the compound of alkyl, alkenyl, or alkynyl is branched or unbranched, unsubstituted or substituted,
or a salt thereof.

In an embodiment, the compound administered has the structure

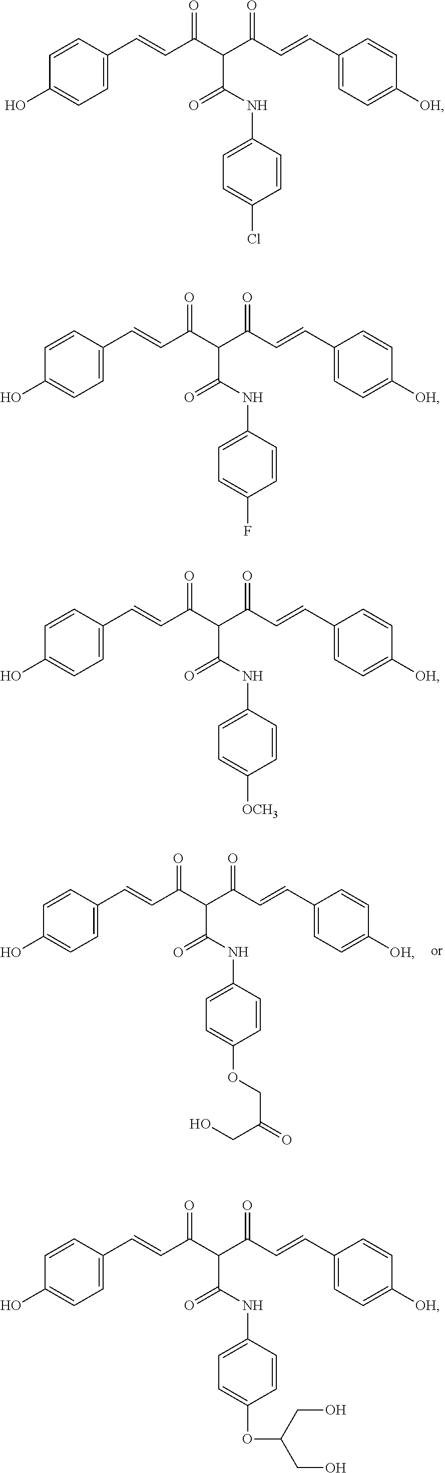

or a salt thereof.

In an embodiment, the compound administered has the structure

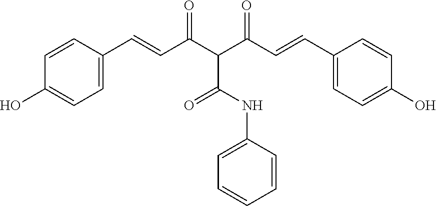 [SMILES: O=C(/C=C/c1ccc(O)cc1)C(C(=O)/C=C/c1ccc(O)cc1)C(=O)Nc1ccccc1]

or a salt thereof.

In an embodiment, the wound is a skin wound.

In an embodiment, the compound is administered to the subject topically.

In an embodiment, the compound is in a carrier which comprises a concentration of the compound.

In an embodiment, the concentration of the compound in the carrier is less than about 5% (w/w).

In an embodiment, the concentration of the compound in the carrier is between about 0.01% and about 3% (w/w).

In an embodiment, the concentration of the compound in the carrier is less than about 3% (w/w).

In an embodiment, the concentration of the compound in the carrier is about 1.5% (w/w).

In an embodiment, the concentration of the compound in the carrier is about 1% (w/w).

In an embodiment, the concentration of the compound in the carrier is about 0.5% (w/w).

In an embodiment, the concentration of the compound in the carrier is between about 0.01% and about 3% (w/w).

In an embodiment, the concentration of the compound in the carrier is about 0.01%, about 0.05%, about 0.1%, about 0.15%, about 0.2%, about 0.25%, about 0.3%, about 0.35%, about 0.4%, about 0.45% or about 0.5% (w/w).

In an embodiment, the concentration of the compound in the carrier is about 0.3% (w/w).

In an embodiment, the concentration of the compound in the carrier is about 0.25% (w/w).

In an embodiment, the concentration of the compound in the carrier is about 0.1% (w/w).

In an embodiment, the carrier is petrolatum jelly.

In an embodiment, the compound is administered to the subject orally.

In an embodiment, the amount of the compound administered is between about 0.1 and about 15.0 mg/kg body weight of the subject/day.

In an embodiment, the subject is a human subject.

In an embodiment, the amount of the compound administered to the human subject is between about 10 mg and about 1000 mg.

In an embodiment, the amount of the compound administered is about 4.0 mg/kg body weight of the subject/day.

In an embodiment, the compound is administered to the subject once per day for as period of 7 days.

In an embodiment, the wound is a skin wound.

In an embodiment, the compound is administered to the subject after the skin wound has begun to heal.

In an embodiment, the skin wound is a cut in the skin, an abrasion of the skin, a puncture of the skin, a burn, contact dermatitis, or an insect bite.

In an embodiment, the skin wound is itching.

In an embodiment, the skin wound is an insect bite.

In an embodiment, the insect is a mosquito, chigger, bed bug, horse fly, or sand fly.

A burn may be a heat burn, a chemical burn, or a radiation burn. A radiation burn may be, e.g., a burn caused by ultraviolet light, such as a sun burn. In some embodiments, a bite may be an insect bite or an arachnid bite. In some embodiments, the insect is a hornet, a wasp, a bee, an ant, a chigger, a bed bug, a mosquito, a sand fly, a horse fly (i.e., a biting insect in the family Tabanidae), a dog fly (i.e., a biting insect in the family Muscidae) or a black fly (i.e., a biting insect in the family Simuliidae). In some embodiments, the arachnid is a mite, flea, tick, scorpion or spider.

In an embodiment, the skin wound is contact dermatitis.

In an embodiment, the contact dermatitis is caused by contact with poison ivy, poison oak, or poison sumac.

Contact dermatitis may be caused by contact with an allergen or an irritant. In some embodiments, contact dermatitis is caused by contact with a poisonous plant. Non-limiting examples of plants that may cause contact dermatitis are those of the Toxicodendron genus, such as poison ivy, poison oak and poison sumac. In some embodiments, contact dermatitis is caused by exposure to a metal, such as nickel. In some embodiments, contact dermatitis is caused by contact with an irritant. Non-limiting examples of irritants that may cause contact dermatitis are detergents and other cleaning products.

A skin wound may be caused by a chemical or biological weapon.

A non-limiting example of a chemical weapon that may cause a skin wound is mustard gas. A non-limiting example of a biological weapon that may cause a skin wound is weaponized B. anthracis (e.g. B. anthracis spores) In an embodiment, the wound is in the oral cavity of the subject.

In an embodiment, the compound is solubilized in a non-toxic organic solubilizing agent.

In an embodiment, the non-toxic organic solubilizing agent is N-methylglucamine.

In an embodiment, the wound is periodontitis, gingivitis, root caries, a canker sore, mucositis, pemphigoid, lichen planus, ulcer or a blistering lesion.

In an embodiment, the method wherein the accelerating of the healing of the wound comprises increasing levels of newly synthesized collagen.

In an embodiment, the method wherein the wound is a skin wound.

In an embodiment, the method wherein the levels of newly synthesized collagen in the wounded skin are increased.

In an embodiment, the method wherein the wound is a skin wound the levels of newly synthesized collagen in the wounded skin are increased.

In an embodiment, the method wherein the subject is diabetic.

In an embodiment, the method wherein the subject is hyperglycemic.

In an embodiment, the compounds of the present invention for use in accelerating the healing of a wound in a subject having a wound.

In some embodiments, including those in which a compound is administered to treat a wound in the oral cavity of a subject, the compound is solubilized in a non-toxic organic solubilizing agent. A non-limiting example of a non-toxic organic solubilizing agent is N-methylglucamine, which is also known as “meglumine”. Therefore, aspects of the invention provide a mouthrinse that comprises a compound of the invention and a non-toxic organic solubilizing agent such as meglumine.

The advantages of using a solubilizing agent such as meglumine include:

1) Meglumine is presumed safe for daily oral use as a topical mouthrinse and, if inadvertently swallowed, should also be safe because it has been used I.V. in the past, to administer other types of agents in humans.
2) A formulation comprising meglumine and a compound of the invention, unlike other popular mouthrinses (e.g., Listerine), does not require alcohol; alcohol-containing mouthrinses have been reported to increase the risk for oral cancer.
3) A formulation comprising meglumine is pleasant tasting since it is a derivative of glucose, namely a glycitol, and would not be metabolized like glucose.

In some embodiments, a compound of the invention is solubilized in meglumine as a mouthrinse (i.e., mouthwash) formulation, and the concentration of the compound in the formulation is effective to suppress MMPs activity and cytokine levels in the oral cavity.

In preferred embodiments, mouthrinse formulations that comprise a compound of the invention do not cause tooth staining.

Aspects of the invention provide toothpaste which comprises a compound of the invention.

This invention provides a pharmaceutical composition comprising a pharmaceutically acceptable carrier and of the above compounds.

This invention also provides a method of inhibiting the activity and/or levels of a matrix metalloproteinase (MMP) comprising contacting the matrix metalloproteinase or a cell producing an MMP or MMPs with any one of the above compounds so as to inhibit the activity of a matrix metalloproteinase.

In an embodiment, the matrix metalloproteinase is MMP-1, MMP-2, MMP-3, MMP-7, MMP-8, MMP-9, MMP-12, MMP-13, or MMP-14.

This invention further provides a method of inhibiting the production of a cytokine in a population of mammalian cells comprising contacting the population of cells with any one of the above compounds so as to inhibit production of a cytokine.

In an embodiment, the population of cells is a population of human cells.

In another embodiment, the cytokine is TNF-α, IL-13, MCP-1, IL-8, or IL-6.

In yet another embodiment, the production of a cytokine is induced by an endotoxin, lipopolysaccharide (LPS), a hormone, a cholesterol complex, or an inflammatory mediator, including but not limited to nitric oxide, and reactive oxygen species.

In another embodiment, the production of a cytokine is induced by a factor that increases cytokine levels in a diabetic subject, such as a non-enzymatic glycated protein or an Advanced Glycation End-product (AGE).

This invention yet further provides a method of inhibiting the production of a growth factor in a population of mammalian cells comprising contacting the population of cells with the any one of the above compounds so as to inhibit production of a growth factor.

In an embodiment, the growth factor is VEGF, PDGF, TGF-β, or MIP1α.

This invention provides a method of inhibiting NFκ-B activation in a population of cells comprising contacting the population of cells with the any one of the above compounds so as to inhibit NFκ-B activation.

In an embodiment, the population of cells is a population of human cells.

It is understood that the structures described in the embodiments of the methods hereinabove can be the same as the structures of the compounds described hereinabove.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### Terms

As used herein, and unless stated otherwise, each of the following terms shall have the definition set forth below.

As used herein, “about” in the context of a numerical value or range means±10% of the numerical value or range recited or claimed, unless the context requires a more limited range.

As used herein, “wound” encompasses any injury in which an external surface, internal mucosa, oral lining or any epithelial tissue of a subject is torn, pierced, cut, abraded or otherwise broken, and any disruption of an external surface, internal mucosa, oral lining or any epithelial tissue of a subject which results from an injury, an infection, from direct contact with an allergen or irritant, or from an autoimmune disease. A non-limiting example of an autoimmune disease is pemphigoid.

As used herein, “skin wound” encompasses any injury in which the skin of a subject is torn, pierced, cut, or otherwise broken, and any disruption of the skin which results from an injury, an infection, from direct contact with an allergen or irritant, or from an autoimmune disease. Examples of skin wounds include but are not limited to cuts, abrasions, punctures, blisters, boils, wheals, burns, rashes, contact dermatitis, bites and psoriasis.

As used herein, “contact dermatitis” means a skin reaction resulting from exposure to an allergen (allergic contact dermatitis) or an irritant (irritant contact dermatitis). Phototoxic dermatitis may occur when an allergen or irritant is activated by sunlight.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n-1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. Where an aryl group is polycyclic, at least 2 aromatic rings are adjacent, i.e. share one side. For example, polycyclic aryl groups do not include moieties containing a tetracycline structure.

Further, the use of the term “polycyclic” is not limited to aryl groups. The term “polycyclic” as used herein may also refer to unsaturated or partially unsaturated multiple fused ring structures. However, the term “polycyclic” as used herein in any context excludes the tetracycline structure.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The term “substituted” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituents include the functional groups described above, and, in particular, halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, abbreviations are defined as follows:
IL=interleukin
MCP=monocyte chemoattractant protein
TNF=tumor necrosis factor
VEGF=vascular endothelial growth factor
MMP=matrix metalloproteinase
LPS=lipopolysaccharide
HPLC=high-performance liquid chromatography

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

Various examples of “curcumin homologues” or “chemically modified curcumins” are described in PCT International Application Publication No. WO 2010/132815, the contents of which are hereby incorporated by reference. Curcumin homologues have improved biological activity compared to curcumin, however some curcumin homologues show toxicity in subjects at high doses. Compound 4 is a curcumin homologue. “Compound 4 homologues” are a subclass of curcumin homologues that are homologues of Compound 4 which have improved biological activity compared to other curcumin homologues such as, for example, Compounds 2 and 3 which are not Compound 4 homologues. Compound 4 homologues are structurally defined hereinabove at the beginning of this Detailed Description of the Invention.

Without wishing to be bound by any scientific theory, the improved biological activity of Compound 4 and its homologues in the subject application may be attributed in part to their ability to access and bind zinc ions and an enhanced solubility. The improved biological activity of Compound 4 and its homologues may also be attributed to their improved ability to bind to the zinc/calcium-based catalytic site in an MMP enzyme compared to another curcumin homologue which is not a Compound 4 homologue. This invention describes that the enhancement of zinc binding and/or MMP catalytic site affinity through the installation of electron-withdrawing and electron-donating groups at strategic locations, namely the C-4 carbon and the aryl rings, on the curcumin skeleton and results in the enhancement of biological activity, including inhibition of MMP activity, NF_{K}B activation, and cytokine production. Additionally, Compound 4 and its homologues are particularly well tolerated in animal and human subjects. PCT International Application Publication No. WO 2010/132815, discusses curcumin homologues, and describes methods of synthesis of curcumin homologues. The entire contents of PCT International Application Publication No. WO 2010/132815 are hereby incorporated by reference.

The compounds of the instant invention may be in a salt form. As used herein, a “salt” is the salt of the instant compounds which has been modified by making acid or base salts of the compounds. Acidic substances can form salts with acceptable bases, including, but not limited to, lysine, arginine, and the like. In the case of compounds administered to a subject, eg. a human, the salt is pharmaceutically acceptable. Examples of pharmaceutically acceptable salts include, but are not limited to, mineral or organic acid salts formed at basic residues such as amino groups; alkali or organic base salts formed at acidic residues such as phenols, carboxylic acids, and carbons having at least 1 acidic hydrogen atom adjacent to a carbonyl. Where acid salts are formed, such salts can be made using an organic or inorganic acid. Such acid salts include, but are not limited to, chlorides, bromides, sulfates, nitrates, phosphates, sulfonates, formates, tartrates, maleates, malates, citrates, benzoates, salicylates, ascorbates, and the like. Because the compounds of the subject invention also possess carbons having at least 1 acidic hydrogen atom adjacent to a carbonyl, enolate salts may be formed by reaction with a suitable base. Suitable bases include, but are not limited, to inorganic bases, such as alkali and alkaline earth metal hydroxides; and organic bases, including, but not limited to, ammonia, alkyl amines, amino alcohols, amino sugars, amino acids, such as glycine, histidine, and lysine, and alkali metal amides, such as lithium diisopropylamide. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds and compositions of this invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other compounds also used to treat rheumatoid arthritis (RA), osteoarthritis (OA), Alzheimer's Disease (AD), metastases, periodontal disease, such as periodontitis, angiogenesis, emphysema, acute respiratory distress syndrome, multiple sclerosis, cardiovascular disease, such as atherosclerosis, myocardial infarction, arterial restenosis after angioplasty and aneurysm development; inflammatory disorders, including neuroinflammation and inflammatory bowel disease; many types of cancer, including breast cancer, skin cancer, including, but not limited to, melanoma, and prostate cancer; diabetes, stroke, peripheral neuropathy, brain trauma, pancreatitis, and skin disorders, including, but not limited to, wounds, including ulcers of the skin, accelerated aging, and inflammatory diseases of the skin, such as psoriasis, acne and rosacea; bone diseases including, but not limited to, osteoperosis. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

A compound of the present invention may be administered to a subject topically in a carrier. In some embodiments, a compound of the invention is administered in a formulation which also comprises petrolatum jelly. It will be understood that compositions which comprise a compound of the invention and a carrier may be formulated by a variety of methods. For example, a finely-ground sample (0.1 g) of a compound of the invention may be i) added to petrolatum jelly (9 g) and ii) the mixture may be homogenized for 15 minutes at 25° C. then iii) allowed to settle for 5 minutes. Steps ii) and iii) may be repeated multiple times until a homogeneous product having a cream-like consistency is obtained. Such a composition may then be used in topical application to wounds.

The improved compounds and compositions of the subject invention are useful for the treatment of skin disorders, including, but not limited to, wounds, including ulcers of the skin, and inflammatory diseases of the skin.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.); and PCT International Application Publication No. WO 2010/132815. All of the aforementioned publications are incorporated by reference herein.

The Compounds such as Compound 2 (a chemically-modified curcumin) may be relatively insoluble in water. Such compounds may be solubilized in a safe organic solubilizing agent, such as meglumine (ie., N-methyl glucamine which is a deoxy(methylamino) glucitol, a derivative of glucose) to solubilize such compounds to improve their efficacy systemically, e.g. by swallowing a teaspoon of a composition comprising a compound of the invention and meglumine qd or even by I.V. injection. Compositions comprising a compound of the invention and meglumine may be used to formulate a therapeutic mouthrinse which is non-toxic, non-alcohol based (because mouthrinses, e.g. Listerine, which contain contain alcohol have been reported to increase the risk of oral cancer), non cariogenic (no sugar content), pleasant tasting (contains an amino-glucitiol sorbitol derivative), and able to suppress MMPs and pro-inflammatory cytokines in the mouth. In some embodiments, the amount of a compound of the invention that is in a mouthrinse is effective to suppress an MMP or pro-inflammatory cytokine in the saliva, an orifice of the periodontal lesion/pocket, or the gingiva. In some embodiments, the amount of a compound of the invention that is in a mouthrinse is effective to treat one or more oral conditions which may include but are not limited toroot caries (particularly in elderly subjects), periodontal disease, mucositis, pemphigoid, lichen planus, and other oral blistering and ulcerative diseases (e.g., aphthous ulcers, and ulcers caused by herpes simplex), and impaired wound healing such as in diabetic subjects.

The compounds and compositions of the subject invention, like curcumin and other curcumin analogues, are useful for treating rheumatoid arthritis (RA), osteoarthritis (OA), metastases, periodontal disease, such as periodontitis, angiogenesis, emphysema, acute respiratory distress syndrome, multiple sclerosis, cardiovascular disease, such as atherosclerosis, myocardial infarction, arterial restenosis after angioplasty and aneurysm development; inflammatory disorders, including neuroinflammation and inflammatory bowel disease; many types of cancer, including breast cancer, skin cancer, including, but not limited to, melanoma, and prostate cancer; diabetes, stroke, peripheral neuropathy, brain trauma, and pancreatitis; bone diseases including, but not limited to, osteoporosis.

Variations on the following general synthetic methods will be readily apparent to those skilled in the art and are deemed to be within the scope of the present invention (47).

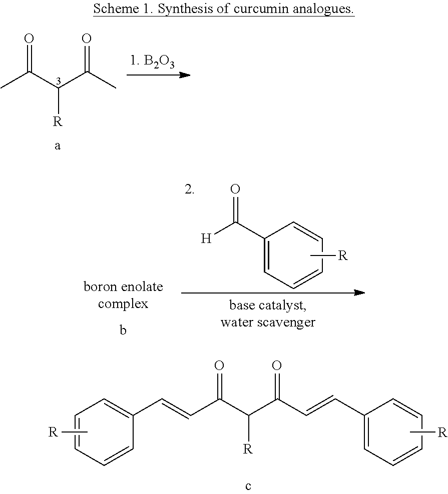

The synthesis of the curcumin analogues of the present invention can be carried out according to general scheme 1. The R groups designate any number of generic substituents.

The starting material is provided by 2,4-pentanedione, which is substituted at the 3-carbon (see compound a). The desired substituted 2,4-pentanedione may be purchased from commercial sources or it may be synthesized using conventional functional group transformations well-known in the chemical arts, for example, those set forth in Organic Synthesis, Michael B. Smith, (McGraw-Hill) Second ed. (2001) and March's Advanced Organic Chemistry: Reactions, Mechanisms, and Structure, Michael B. Smith and Jerry March, (Wiley) Sixth ed. (2007), and specifically by Bingham and Tyman (45) and in the case of 3-aryl-aminocarbonyl compounds by Dieckman, Hoppe and Stein (46), the contents of which are hereby incorporated by reference. 2,4-pentanedione a is reacted with boron trioxide to form boron enolate complex b.

Boron enolate complex b is a complex formed by coordination of the enolate of compound a with boron. It is understood by those having ordinary skill in the art that the number of compound a enolates that may coordinate to boron as well as the coordination mode, i.e. monodentate versus bidentate, are variable so long as reaction, such as Knoevenagel condensation, at the C-3 carbon of the 2,4-pentanedione is suppressed.

Boron enolate complex b is then exposed to a benzaldehyde compound in the presence of a base catalyst and a water scavenger to form curcumin analogue c via aldol condensation. The ordinarily skilled artisan will appreciate that the benzaldehyde may possess various substituents on the phenyl ring so long as reactivity at the aldehyde position is not hindered. Substituted benzaldehyde compounds may be purchased from commercial sources or readily synthesized using aryl substitution chemistry that is well-known in the art. Suitable base catalysts for the aldol step include, but are not limited to, secondary amines, such as n-butylamine and n-butylamine acetate, and tertiary amines. Suitable water scavengers include, but are not limited to, alkyl borates, such as trimethyl borate, alkyl phosphates, and mixtures thereof. Other suitable reaction parameters have also been described by Krackov and Bellis in U.S. Pat. No. 5,679,864, the content of which is hereby incorporated by reference. Additional methods of

All combinations of the various elements described herein are within the scope of the invention.

Herein, where chemical substituents are disclosed in the alternative, it is intended that each such substituent can be used or combined with one or more other substituents disclosed in the alternative.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1

### Synthesis of Chemically Modified Curcumin Analogues

The synthetic methods for the preparation of Compound 4 and several homologues of curcumin, confirmation of their structure, and a summary of the in vitro screening against a series of MMPs is described hereinbelow in Table 1.

### Methods:

All reagents and solvents employed in the experimental work were of reagent grade and were used as such unless otherwise specified. Melting points were taken in a Thomas-Hoover open-capillary melting-point apparatus and are uncorrected. ¹H NMR spectra were recorded on a Varian Gemini 300 spectrometer. Samples prepared for NMR analysis were dissolved in CDCl3 or DMSO-d6. Chemical shifts are reported in parts per million (ppm) relative to TMS. Mass spectra were recorded on either a Thermo Electron DSQ GC/MS equipped with a solid probe inlet and EI ionization or an Agilent 1100LC (API-ES)/MSD-VL(m/z=50-1500) using electrospray ionization. Thin-layer chromatography (TLC) was performed on silica gel sheets (Tiedel-deHaen, Sleeze, Germany). After appropriate purification all new products showed a single spot on TLC analysis in the following solvent system: 25% EtOAc in hexanes. Components were visualized by UV light (l=254 nm). Flash column chromatographic separations were carried out on 60A ° (230-400 mesh) silica gel (TSI Chemical Co., Cambridge, Mass.). All experiments dealing with moisture or air-sensitive compounds were conducted under dry nitrogen.

| Chemicals | Grade | Supplier |
|---|---|---|
| 2,4- | 99% | Alfa Aesar |
| Pentanedione(Acetoacetone) | | |
| Magnesium chloride | Anhydrous | Sigma-Aldrich |
| Pyridine | 99.0% min | Alfa Aesar |
| Methyl Chlorofomate | 97% | Acros Organics |
| Phenyl Isocyanate | 98+% | Sigma-Aldrich |
| Boron Oxide | 99.98%, metals | Alfa Aesar |
| | basis | |
| Vanillin | 99% | Sigma-Aldrich |
| 4-Hydroxybenzaldehyde | 96% | Sigma-Aldrich |
| Trimethyl Borate | 99% | Alfa Aesar |
| Methyl Alcohol (Methanol) | 99.8%, | Sigma-Aldrich |
| | anhydrous | |
| Methylene chloride | 99.9% | Fisher Scientific |
| Ethyl acetate | 99.9% | Fisher Scientific |
| Sodium Sulfate | Anhydrous | EMD |

### Reagents:

### General Procedures for the Synthesis of Curcumin Analogues:

### Step A. 3-Acylated 2,4-Pentanediones

Acetylacetone(1.00 g, 10 mmol) was added to a suspension of magnesium chloride(1.35 g, 1.2 eq) in 20 mL methylene chloride, followed by pyridine(2.13 mL, 2.5 eq), and the mixture was stirred at 0° C. for 1 h, then an alkyl chloroformate or a phenyl isocyanate(1.0 eq) was added dropwise to the mixture at 0° C. The reaction mixture was allowed to warm up to room temperature during 8 hours, and then was poured into 3N aqueous HCl solution(10 mL) and extracted with methylene chloride(20 mL). The organic layer was washed with brine (20 mL), dried over sodium sulfate and the solvent was removed under vacuo. The product was then distilled or recrystallized from an appropriate solvent depending on the physical state at RT.
1. 3-(Methoxycarbonyl)acetylacetone: colorless liquid, 68.1% yield. Distilled at 45° C., 0.5 mmHg. ESI (−ve) MS m/z 157.1 [M-H]⁻. ¹H NMR (DMSO-d₆, 300 MHz): δ 2.999 (s, 6H), 3.730 (s, 3H), 17.760 (s, 1H).
2. 3-(Ethoxycarbonyl)acetylacetone: colorless liquid, 60.5% yield. Distilled at 60° C., 1.4 mmHg. ESI (−ve) MS m/z 171.2 [M-H]⁻. 1H NMR (DMSO-d, 300 MHz): δ 1.253 (t, 3H) 2.303 (s, 6H), 4.164-4.236 (m, 2H), 17.747 (s, 1H).
3. 3-(Phenylaminocarbonyl)acetylacetone: white solid, 72.0% yield. mp 118-119° C. ESI (−ve) MS m/z 218.1 [M-H]⁻. ¹H NMR (DMSO-d₆, 300 MHz): δ 2.154 (s, 6H), 7.084 (t, 1H), 7.324 (t, 2H), 7.640 (d, J=8.1 Hz, 2H), 10.355 (s, 1H), 16.463 (s, 1H).

### Step B. Curcumin Analogues

Acetylacetone or a 3-substituted acetylacetone (10 mmol) and boron oxide (0.49 g, 7 mmol, 0.7 eq) were placed in a 50 mL flask and heated to 120° C. for 5 min to form a pale-yellow suspension. The appropriate aldehyde (20 mmol, 2.0 eq) and trimethyl borate (4.16 g, 40 mmol, 4.0 eq) were dissolved in ethyl acetate (10 mL) and gradually added to reaction mixture. Thereafter, with stirring, 0.05 mL of butylamine and 0.2 mL of butylammonium acetate in dimethylformamide solution (0.136 g/mL) were added. After 1 hour, a red-colored precipitate started to form. The whole reaction mixture was stirred at room temperature for 48 hours. The precipitate was filtered and dried, then dissolved in methanol (50 mL) and boiled for 30 min at 60° C. Methanol was removed by rotary evaporation and the crystalline crude product was purified by recrystallization from dichloromethane (20 mL) and methanol (20 mL).
1. Curcumin (Compound 1): 1,7-Bis(4-hydroxy-3-methoxyphenyl)hepta-1E,6E-dien-3,5-dione derived from vanillin and acetylacetone: orange crystals, 77.0% yield. mp 175-176° C. ESI (−ve) MS m/z 367.2 [M-H]⁻. 1H NMR (DMSO-d₆, 300 MHz): δ 3.335 (s, 6H), 5.556 (s, 1H), 6.256 (d, J=15.6 Hz, 2H), 6.320 (d, J=7.8 Hz, 2H), 6.647 (d, J=8.4 Hz, 1H), 6.653 (d, J=8.1 Hz, 1H), 6.822 (d, J=2.1 Hz, 2H), 7.044 (d, J=15.9 Hz, 2H), 9.162 (s. 2H), 15.889 (s, 1H);
2. 4-Methoxycarbonylcurcumin: 1,7-Bis(4-hydroxy-3-methoxyphenyl)-4-methoxycarbonylhepta-1E,6E-dien-3,5-dione (Compound 2): derived from vanillin and 3-methoxycarbonyl-2,4-pentandione: yellow crystals, 72.0% yield. mp 175-176° C. ESI (−ve) MS m/z 425.2 [M-H]⁻. ¹H NMR (DMSO-d₆, 300 MHz): δ 3.819 (s, 3H), 3.903 (s. 6H), 6.834 (d, J=7.2 Hz, 2H), 7.019 (d, J=15.6, 2H), 7.189 (d, J=8.1 Hz, 1H), 7.195 (d, J=8.4 Hz, 1H), 7.272 (d, J=1.5 Hz, 2H), 7.722 (d, J=15.6 Hz, 2H), 9.840 (s. 2H), 18.294 (s, 1H);
3. 4-Ethoxycarbonylcurcumin. (Compound 5): 1,7-Bis(4-hydroxyphenyl)-4-methoxycarbonylhepta-1E,6E-dien-3,5-dione derived from 4-hydroxybenzaldehyde and 3-methoxycarbonyl-2,4-pentandione: yellow crystals, 49.2% yield. mp 214-216° C. ESI (−ve) MS m/z 365.0 [M-H]⁻. ¹H NMR (DMSO-d₆, 300 MHz): δ 3.900 (s. 3H), 6.831 (d, J=8.4 Hz, 4H), 6.986 (d, J=15.6 Hz, 2H), 7.576 (d, J=8.7 Hz, 4H), 7.729 (d, J=15.6 Hz, 2H), 10.172 (s. 2H), 18.267 (s, 1H);
4. (Compound 3): 1,7-Bis(4-hydroxy-3-methoxyphenyl)-4-N-phenylaminocarbonylhepta-1E, 6E-dien-3,5-dione derived from vanillin and 3-phenylaminocarbonyl-2,4-pentandione: orange crystals, 46.6% yield. mp 193-194° C. ESI (−ve) MS m/z 486.0 [M-H]⁻. ¹H NMR (DMSO-d₆, 300 MHz): δ 3.701 (s. 6H), 6.730 (d, J=15.6 Hz), 6.790 (d, J=8.1 Hz), 7.082-7.152 (m, 5H), 7.371 (t, 2H), 7.685 (d, J=3.9 Hz, 2H), 7.723 (d, J=3.9 Hz, 2H), 9.788 (s. 2H), 10.586 (s. 1H), 17.556 (s, 1H);
5. Compound 4: 1,7-Bis(4-hydroxyphenyl)-4-N-phenylaminocarbonylhepta-1E, 6E-dien-3,5-dione derived from 4-hydroxybenzaldehyde and 3-phenylaminocarbonyl-2,4-pentandione: yellow crystals, 46.2% yield. mp 220-221° C. ESI (−ve) MS m/z 426.0 [M-H]⁻. ¹H NMR (DMSO-d₆, 300 MHz): δ 6.681 (d, J=15.6 Hz, 4H), 6.787 (d, J=8.7 Hz, 4H), 7.133 (t, 1H), 7.375 (t, 2H), 7.453 (d, J=9.0 Hz, 2H), 7.695 (d, J=6.3 Hz, 2H), 7.723 (d, J=9.0 Hz, 2H), 10.144 (s. 2H), 10.605 (s. 1H), 17.559 (s, 1H);

### Large Scale Procedure for Synthesis of Compound 4

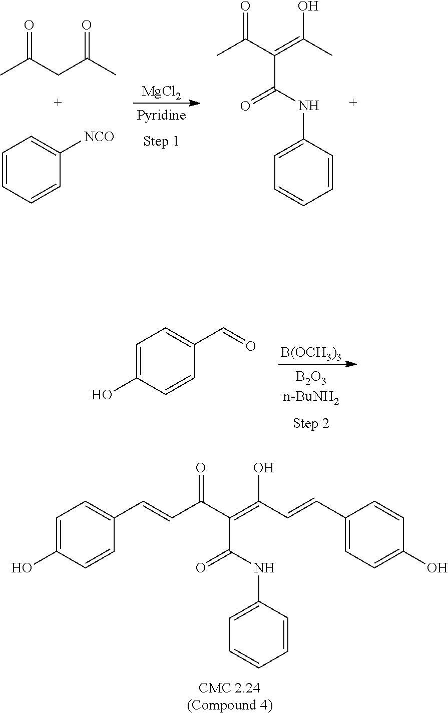

### Step A

Acetylacetone (140 g, 1.40 mol, 1.0 eq) was added to a suspension of anhydrous magnesium chloride (153 g, 1.61 mol, 1.15 eq) in dichloromethane (2.5 L) followed by pyridine (188 g, 2.38 mol, 1.7 eq) while stirring at −5° C. After 45 minutes, phenyl isocyanate (142 mL, 1.4 mol, 1.0 eq) was added to the reaction mixture and stirring continued for 2 hours at −5° C. The reaction was quenched with 5% citric acid (1 L) and extracted with dichloromethane (3×500 mL). The organic layer was concentrated and triturated/recrystallized from 5% acetone in hexanes to obtain the desired product (261 g, 86%).

¹H NMR CDCl₃: 18.564, 16.422 (s, 1H, enolate OH), 12.024 (s, 1H, NH), 7.55 (m, 2H), 7.38 (m, 2H), 7.17 (m, 1H), 2.52 (s, 6H). mp 72-73° C.

### Step B

N-Phenylaminocarbonyl-pentane-2,4-dione (35.14 g, 160.5 mmol, 1.0 eq) and finely powdered boron trioxide (6.74 g, 96.3 mmol, 0.60 eq) were finely ground and mixed together then placed into a 1 L flask. This was heated to 145° C. and stirred vigorously for 1 hr to form the boron complex. The temperature was lowered to 65-70° C. when a mixture of 4-hydroxybenzaldehyde (43.1 g, 353 mmol, 2.2 eq) and trimethyl borate (69.9, 641.8 mmol, 4.0 eq) in dry ethyl acetate (450 mL) which was previously stirred for 1 hour at room temperature was added slowly. While stirring and after addition at this temperature, a catalyst of butylamine (0.75 mL) and butylammonium acetate (3.0 mL) in dimethylforamide solution (0.136 g/mL) were added. The reaction mixture was stirred for 2 days at 65-70° C. Afterwards, the reaction was cooled to room temperature and the precipitate was filtered and washed with diethyl ether. The solid was dissolved in 1.5:1 mixture of 1,2-dichloroethane/methanol (300 mL/200 mL) and heated for 30 minutes and then the methanol was distilled. The process was repeated twice and then the solution was concentrated to remove solvent and the product (Compound 4) was recrystallized from 1:1 dichloromethane/methanol to obtain the pure material. (45%, 30.8 g)

Compound 4 ¹H NMR DMSO-d₆: 17.564 (s, 1H, enolate OH), 10.608 (s, 1H, NH), 10.151 (s, 2H, OH), 7.707 (m, 4H), 7.46 (d, 4H), 7.37 (t, 2H), 7.133 (t, 1H), 6.801 (d, 4H), 6.7 (d, 2H). mp 220-221° C.

### Example 2

### Compound 4 is Highly Effective at Inhibiting MMPs and is Well Tolerated

Curcumin (Compound 1) has a long history as an herbal medication and has been recommended at one time or another for the treatment of almost every human ailment from simple wound-healing to cancer (48). However, its lack of solubility in physiological fluid has all but precluded its use as a medicinal substance. There is an identity of the simple 1, 3-diketo array in curcumin with that of the useful drug substance doxycycline (the basis of the drug substances Periostat and Oracea) (4). This is the active-site in doxycycline, at positions 11, 11a, and 12, that can chelate zinc and calcium in MMP enzymes. The compounds of the invention are more effective at inhibiting MMP enzymes than curcumin. Additionally, the compounds of the invention, and in particular, homologues of Compound 4, are well tolerated in animals, and in human subjects. As disclosed herein, curcumin-like substances, containing an additional electron-withdrawing carbonyl-based residue at the 4-position, have enhanced zinc-binding properties. Additionally, Compound 4 and its homologues have an enhanced ability to bind to the zinc/calcium-based catalytic site in MMP enzymes compared to other curcumin homologues which are not Compound 4 homologues.

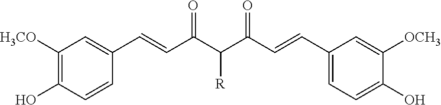

1. (R═H)
2. (R═CO₂R′ (R′═CH₃ or C₂H₅))
3. (R=PhNHCO—)

Initially compounds with an ester function at the 4-position represented by structure 2 were studied. Although these compounds showed useful anti-MMP activity, they were not judged to be active enough or more importantly, soluble enough to be effective medications. With this lead however Compound 3 was synthesized and found to be highly active, but to have some toxicity at elevated levels. The lower homologue 4 (Compound 4) has proved to be the best lead compound. Compound 4 was highly active against a battery of MMPs (see, e.g. Table 1), yet showed virtually no toxic effects at 30 mg/kg body weight in diabetic rats. Its spectrum of activity was wide-ranging and it may be a very useful drug substance.

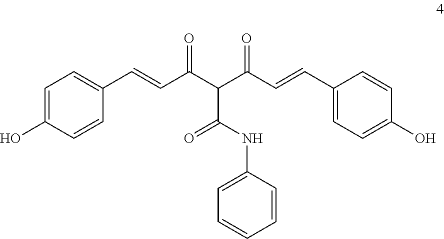 [SMILES: O=C(/C=C/c1ccc(O)cc1)C(C(=O)/C=C/c1ccc(O)cc1)C(=O)Nc1ccccc1]

TABLE 1
In Vitro potency of curcumin and selected chemically-modified curcumins (CMCs) as inhibitors of
nine different human MMPs. In this experiment, a fluorimetric assay of MMP activity was used.

| | | Compounds | | | | | |
|---|---|---|---|---|---|---|---|
| | | 1,10- | Curcumin | CMC2.5 | CMC2.14 | CMC2.23 | CMC2.24 |
| MMPs | | Phen* | (Compound 1) | (Compound 2) | (Compound 5) | (Compound 3) | (Compound 4) |
| Collagenases | MMP-1 | 42.0 ± 1.1 | 85.8 ± 1.8 | 74.0 ± 3.5 | 76.3 ± 6.5 | 68.0 ± 3.2 | 69.8 ± 2.0 |
| | MMP-8 | 31.3 ± 0.5 | 6.8 ± 1.0 | 30.8 ± 1.5 | 20.0 ± 2.0 | 2.5 ± 0.3 | 4.5 ± 0.5 |
| | MMP-13 | 50.0 ± 10.4 | 3.7 ± 0.3 | 28.3 ± 4.4 | 26.7 ± 1.7 | 3.3 ± 0.3 | 2.7 ± 0.7 |
| Gelatinases | MMP-2 | 73.8 ± 1.0 | 5.0 ± 0.7 | 25.3 ± 1.3 | 23.8 ± 0.9 | 6.3 ± 0.9 | 4.8 ± 0.5 |
| | MMP-9 | 45.0 ± 12.6 | 30.0 ± 2.9 | 55.0 ± 17.3 | 43.3 ± 4.4 | 8.7 ± 0.7 | 8.0 ± 0.6 |
| Others | MMP-3 | 77.0 ± 3.2 | 4.7 ± 0.8 | 32.5 ± 2.8 | 28.3 ± 1.0 | 5.3 ± 0.7 | 2.9 ± 0.4 |
| | MMP-7 | 196.8 ± 8.8 | 51.8 ± 2.5 | 48.8 ± 0.5 | 57.5 ± 4.6 | 21.5 ± 1.0 | 5.0 ± 0.7 |
| | MMP-12 | 29.5 ± 1.3 | 2.6 ± 0.2 | 27.8 ± 1.7 | 5.3 ± 0.3 | 4.5 ± 0.5 | 2.0 ± 0.4 |
| | MMP-14 | 43.8 ± 4.2 | 29.5 ± 3.2 | 48.5 ± 4.3 | 40.0 ± 8.4 | 41.3 ± 4.9 | 15.3 ± 3.1 |

*1,10-phenanthroline, a Zinc cation binding agent traditionally used to quench in vitro MMP activity reactions, was used as the positive control for the experiment.
IC₅₀ were measured using a synthetic fluorescent peptide substrate (Mca-Lys-Pro-Leu-Gly-Leu-Dpa-Ala-Arg-NH₂), an excellent substrate for MMPs with cleavage site between Gly and Leu.

### Example 3

### A Novel Chemically Modified Curcumin (Compound 4) Improves Wound-Healing Impaired by Diabetes

### Methods

### Diabetes Induction and Wounding

Diabetes was induced in 12 adult male rats (300-325 g. body weight) by streptozotocin (STZ) injection (70 mg/kg) into the tail vein as previously described (34, 37, 49), and all 15 rats (including three non-diabetic control rats not treated with streptozotocin) were given unlimited access to food and water; all STZ-injected rats exhibited pathologically elevated glucose levels in their urine. Three weeks after inducing diabetes, the back skin of all the rats was shaved and a series of 6 standard wounds per rat, each 6 mm in diameter, was made using a surgical trephine.

### Formulation of Composition for Topical Treatment

A finely-ground sample (0.1 g) of Compound 4 [4-(phenylaminocarbonyl)-1,7-bis(4-hydroxyphenyl)hepta-1e,6e-diene-3,5-dione] was added to petrolatum jelly (9 g) and the mixture was homogenized for 15 minutes at 25° C., then allowed to settle for 5 minutes. This procedure was repeated nine times, producing a homogeneous product having a cream-like consistency. This was then used in topical application to wounds.

### Administration

The following five experimental groups (n=3 rats per group) were established: Non-diabetic control rats (NDC) treated by daily application of white petrolatum jelly (“vehicle”) for 7 days, diabetic rats (D group) treated daily with vehicle alone; diabetics treated by daily topical application of either a 1% (D+1%) or a 3% (D+3%) suspension of Compound 4 in the vehicle; and diabetics treated systemically by daily oral intubation of a 1 ml suspension of Compound 4 (in carboxymethylcellulose) at a dose of 30 mg/kg over the 7-day treatment protocol (D+30 mg/kg). At the end of this time period, the circular wounds were clinically assessed by measuring diameter of the wound in millimeters, blood samples were collected, the rats were sacrificed, and skin samples collected for histological/histochemical, and biochemical assessment as described below.
1) On day 7 after inducing wound healing, the animals were anesthetized and blood samples collected for blood glucose (one Touch Ultra Glucometer; Johnson & Johnson, New Brunswick, N.J.) and Hemoglobin A1c (A1c Now+; Bayer Health Care, Sunnyvale, Calif.) measurements.
2) Photographs were taken for clinical observations to assess wound closure. The % reduction of the wound surface was calculated by measuring the diameter (in millimeters) of each wound before and after the treatment protocol.
3) Wound tissues on day 7 were excised from 3 sites per rat and pooled for biochemical analysis. Each pool of tissue was homogenized, extracted at 4° C. with 5M urea in 50 mM Tris-HCl buffer (pH7.8) containing 0.2M NaCl and 5 mM CaCl2 overnight, then centrifuged for 1 hour at 11,000×g, as described by us previously (50,51). The supernates were then dialyzed against the Tris-HCl, NaCl, CaCl2 buffer, and the proteinases partially purified by ammonium sulfate precipitation and examined for levels of MMP-2 (72 kD gelatinse) and MMP-9 (92 kD gelatinase) by gelatin zymography using denatured type I collagen (gelatin) as the substrate (6).
4) The remaining wounds were assessed histologically & histochemically by Dr. Steve McClain (McClain Labs, Smithtown, N.Y.) as follows: Biopsies of each wound site were taken and included both the wound tissue and surrounding non-wounded tissue. Each biopsy was fixed in 10% neutral buffered formalin for 24 hours, then in 50% ethanol before paraffin sectioning. Each section was stained with H & E and Masson's Trichrome.

### Results

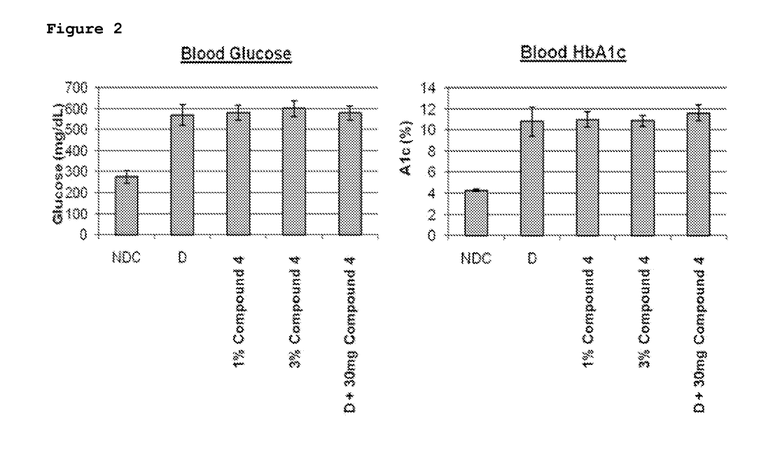

As expected, all four groups of diabetic rats (including vehicle only-treated diabetic animals plus those diabetics treated either topically or systemically with Compound 4) showed dramatically elevated blood glucose & HbAlc levels compared to the non-diabetic control (NDC) rats (FIG. 2); note that all blood samples were collected from non-fasted rats. Moreover, none of the topical or systemic treatments with Compound 4 demonstrated any effect on the severity of hyperglycemia in these diabetics.

However, in spite of this lack of effect on severity of diabetes, all three treatments (two topical and one systemic) dramatically enhanced and accelerated wound healing in vivo. The clinical benefits of these studies are described hereinbelow.

### Example 4

### Clinical Assessment and Measurements

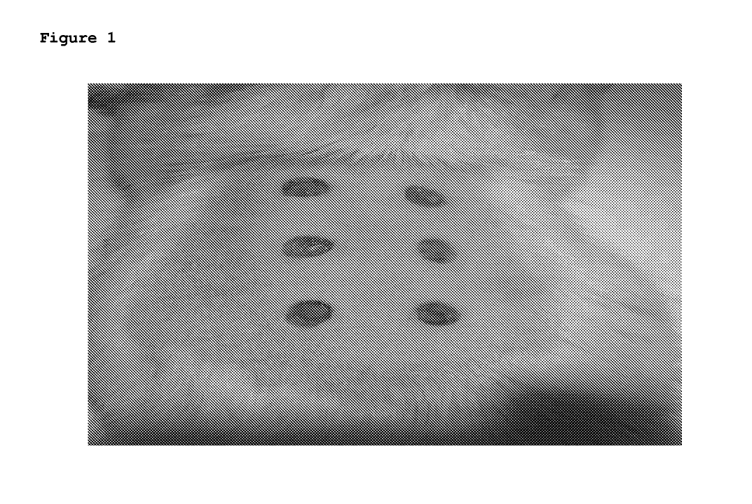

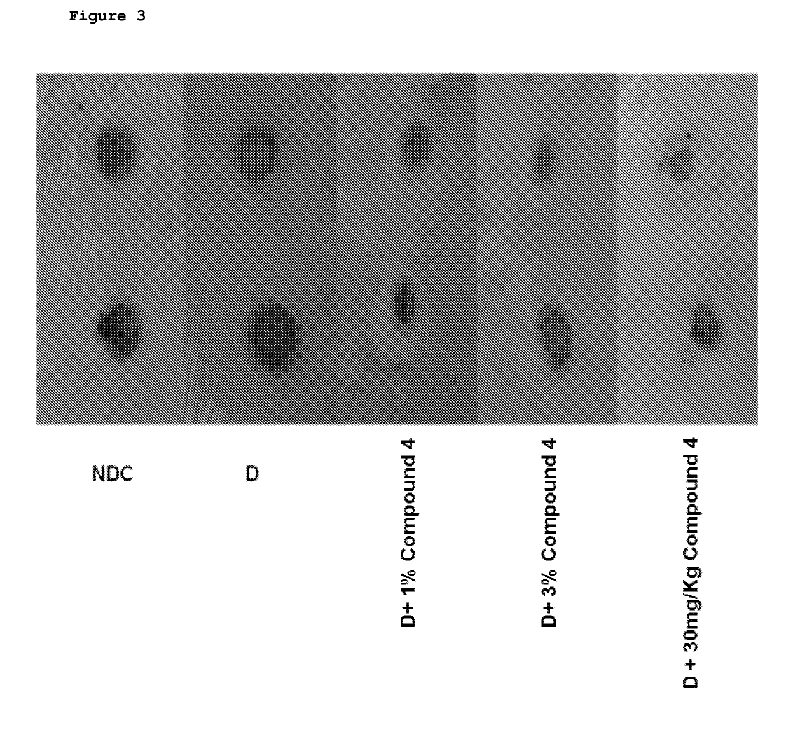

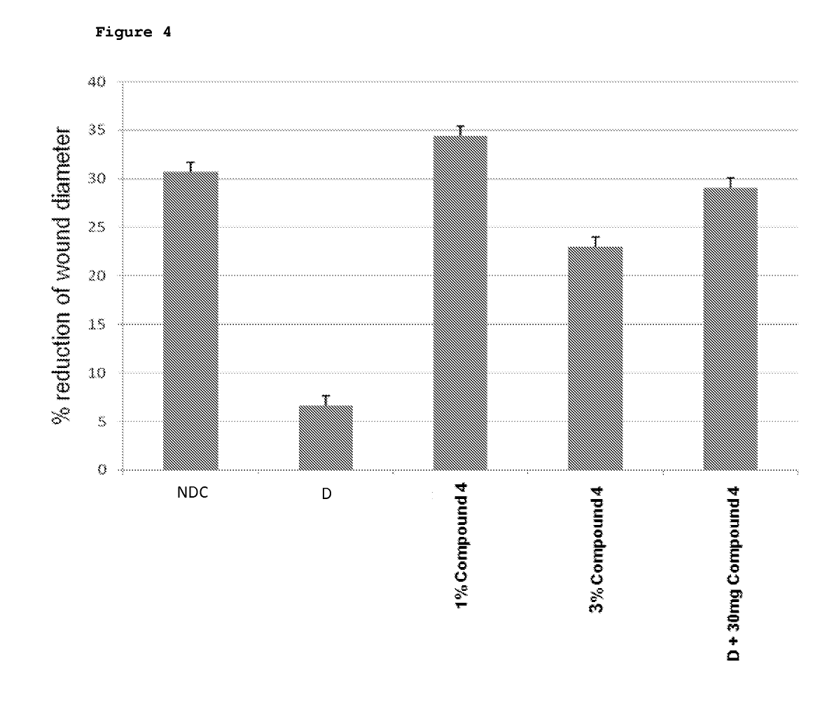

A comparison of the data shown in FIG. 1, which shows a standard series of 6 wounds, 6 mm in diameter, on the backskin of a control non-diabetic or diabetic rat at the time of circular biopsy on day “0”. FIG. 3 shows the clinical appearance of diabetic wounds after 7 days of treatment with Compound 4, indicating that the induction of diabetes (D) appeared to delay/retard wound healing compared to the non-diabetic control rats (NDC). Interestingly, all three Chemically Mofified Curcumin (CMC)-treated groups of diabetic rats appeared to show improved wound healing compared to the vehicle (placebo) treated diabetics. Furthermore, the CMC-treated diabetic rats, particularly those treated topically with 1% Compound 4 showed better wound healing than the non-diabetic control rats (FIG. 3). Quantitative measurements of the diameter of each wound (6 wounds/rat) before and after the 7-day treatment with Compound 4 revealed that the healing exhibited by vehicle-treated diabetic rats (D group) was suppressed by about 80% compared to the healing in the non-diabetic control rats (NDC) (FIG. 4). Based on the data in FIG. 4, which shows the percent reduction of the diameter of the skin wounds over the 7-day protocol for each group of rats, the most effective treatment was the 1% topical Compound 4 as evidenced by an 35% reduction in wound diameter when compared to the 30% reduction in wound diameter for the non-diabetic control group; followed by systemic treatment with Compound 4, which returned wound healing characteristics to that seen in the non-diabetic control group; and the 3% topical application of Compound 4 that exhibited a significant increase in wound healing over that of the untreated diabetic control (D) but failed to equal the wound healing characteristics of the non-diabetic control samples.

### Example 5

### Biochemical Assessment

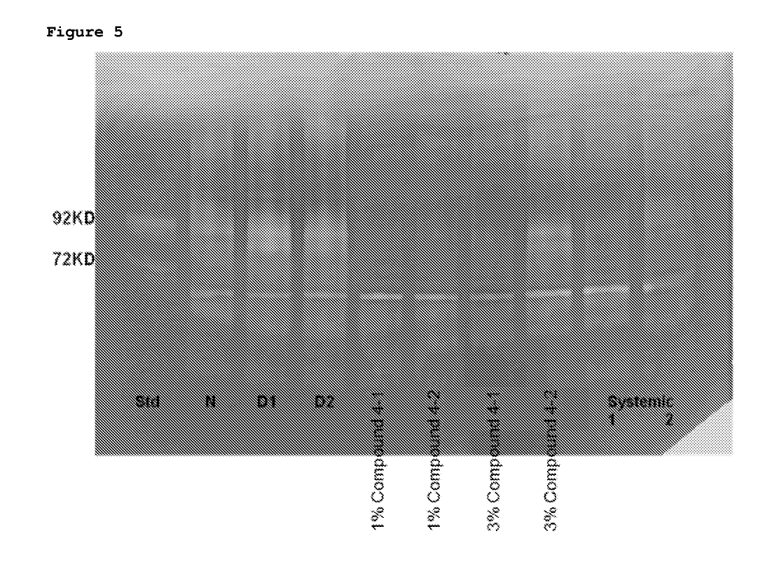

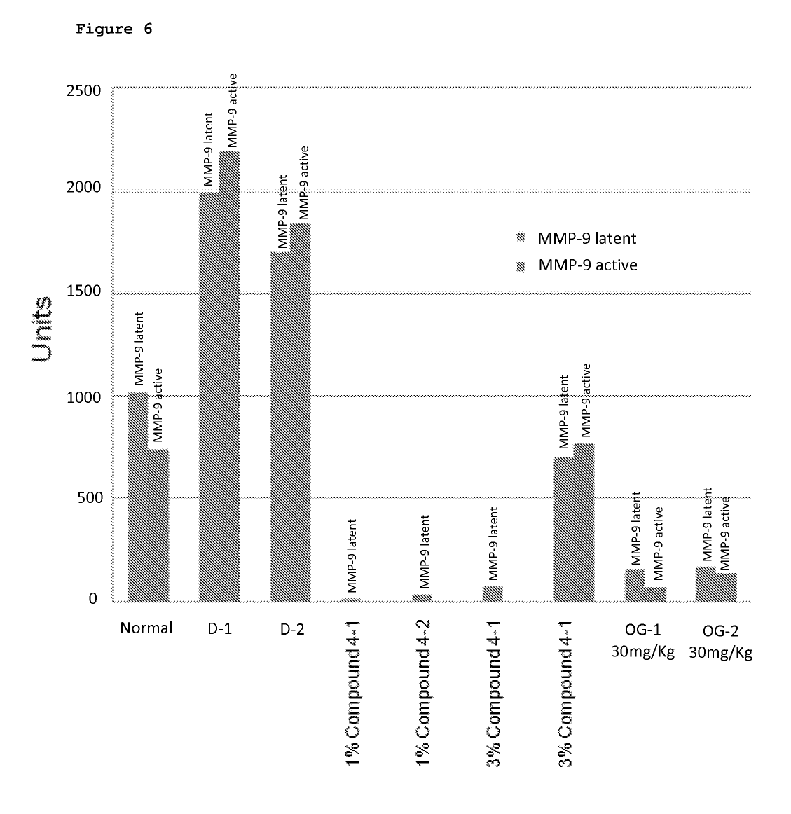

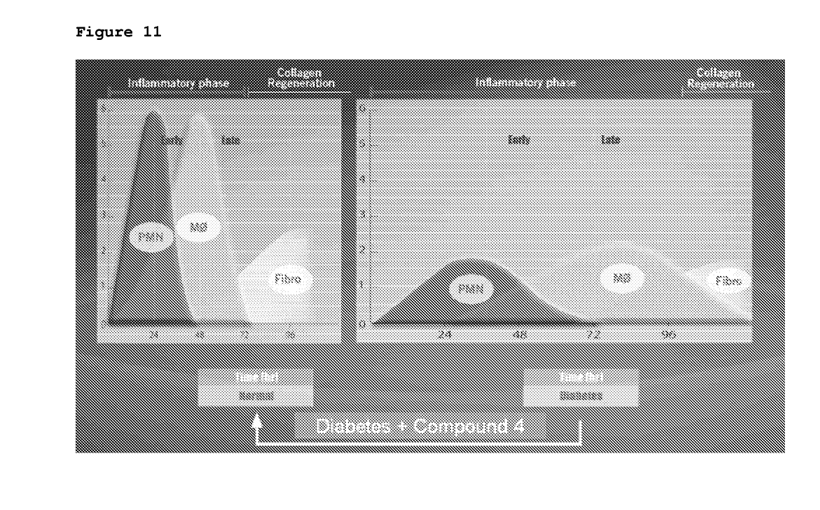

On day 7, after sacrificing the rats, 3 of the 6 wounds per rat were dissected, pooled, and extracted in standard Tris/HCl (pH7.8), NaCl, CaCl2 buffer. The wound extracts for each rat were then partially purified by ammonium sulfate precipitation, and aliquots were examined for MMP-2 (72 kDa gelatinase) and MMP-9 (92 kDa gelatinase) by gelatin zymography using denatured type I collagen or gelatin as substrate as previously described (4-6). Representative data are shown in FIG. 5. Briefly, extracts of skin wounds from each of the 5 groups of rats revealed the presence of active forms of MMP-2 (<72 kDa, compared to 72 kDa standards). Interestingly, the most striking difference among the Compound 4 treated rats was the reduction of 92 kDa gelatinase or pro-MMP-9, as well as reductions in the lower molecular weight activated MMP-9. Clearly, FIG. 5 indicates that inducing diabetes (D1 and D2) increased the level of pro-MMP-9 (and activated MMP-9), a connective tissue-destructive proteinase, when compared to the level seen in the non-diabetic controls (N), and all three treatments with Compound 4 attenuated this diabetic effect. Again, ranking the efficacy of the three treatment regimens, topical 1% Compound 4 was slightly more effective than systemic (oral gavage), which was more effective than topical treatment with 3% Compound 4. The same ranking was seen when the zymograms were scanned densitometrically (FIG. 6). Taken together, and without wishing to be bound by any scientific theory, these findings imply that treatment with 1% Compound 4 is attenuating MMP-9, which is associated with inflammatory cells, better than either 3% topical treatment of Compound 4 or systemic treatment of Compound 4, but treatment with 1% Compound 4 does not suppress, and perhaps even enhances, MMP-2 which is associated with fibroblasts and connective tissue regeneration and remodeling. Thus, treatment with 1% Compound 4 may be accelerating both the resolution of the inflammatory phase of wound healing, and the regeneration of connective tissue (FIG. 11).

### Example 6

### Histological Assessment and Histomorphometric Measurements

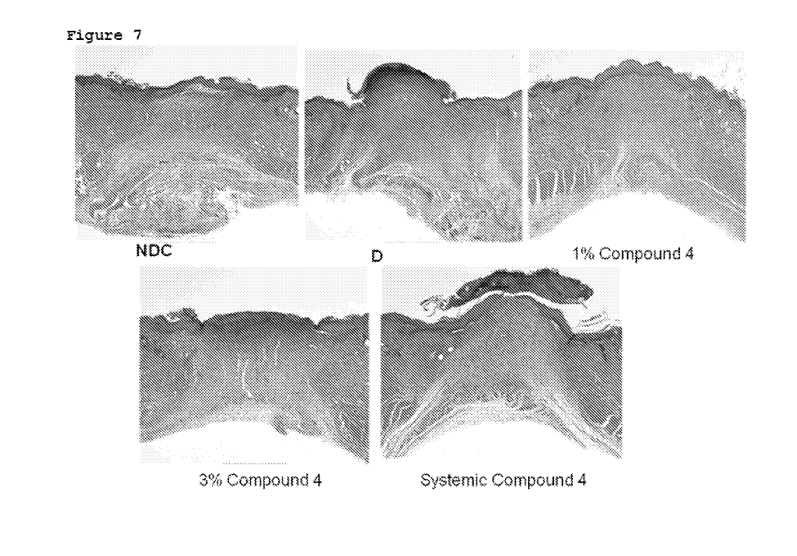

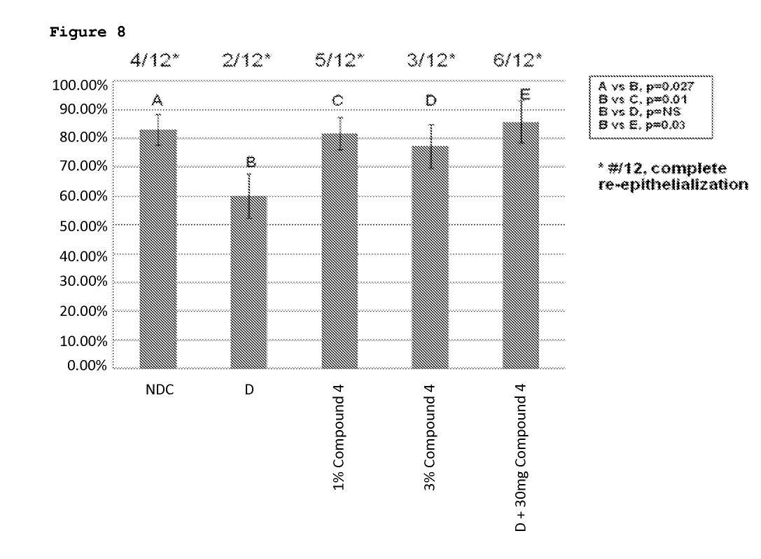

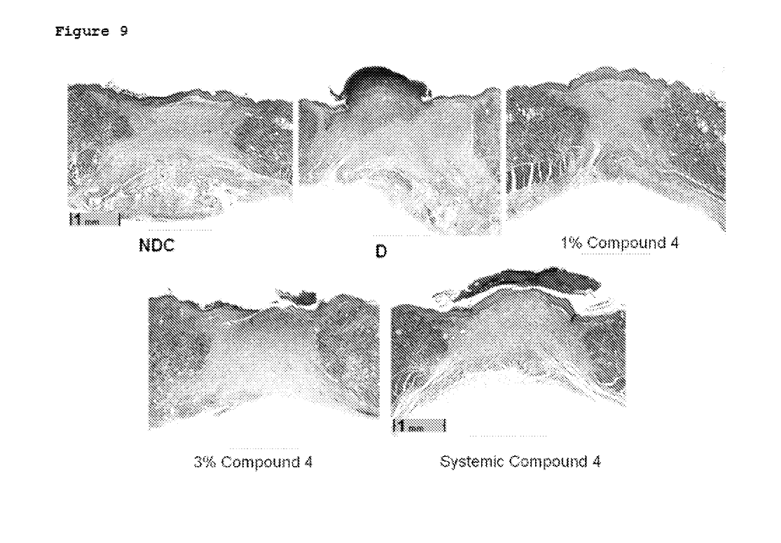

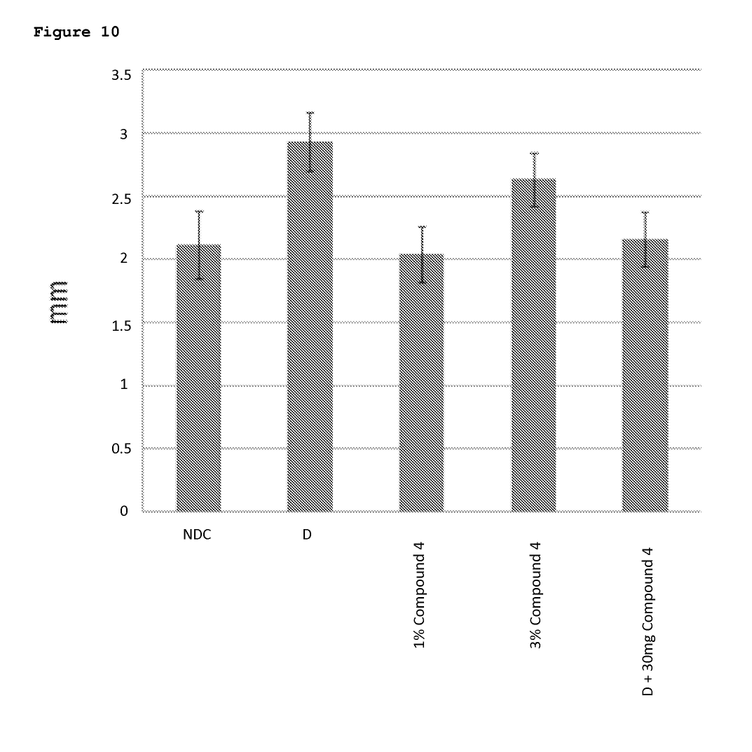

A histological analysis of skin tissue in control and diabetic wound rat models yielded results consistent with the clinical and biochemical data discussed hereinabove. Induction of diabetes (D) suppressed healing of both the epithelium and the underlying connective tissue compared to the normal, non-diabetic control rats (NDC). Furthermore, topical treatment with 1% Compound 4 and systemic Compound 4 were more effective than treatment with topical 3% Compound 4 (FIGS. 7-10). Of interest, the histologic measurement of wound diameter in millimeters, shown in FIG. 10, taken seven days after creating the wounds, exhibited a very similar pattern of change when compared to the clinical measurement of wound healing presented in FIG. 4, and to a lesser extent FIGS. 1-10. These data indicate that Compound 4, when applied topically, and to a lesser extent systemically, is a very potent medication to promote wound healing associated with diabetes.

Compound 4 enhances wound healing in medically-healthy animals and in normal humans as well, and is well tolerated following topical (e.g., 1% Compound 4) or systemic administration.

### Example 7

### Compound 4 Attenuates the Prolonged Inflammatory Response During Diabetes

### Materials & Methods (for Peritoneal Macrophages)

Adult male Sprague-Dawley rats (body weight 300-350 g) were injected through the tail vein with either 0.9% saline (non-diabetic control) or with the same solution containing streptozotocin (70 mg/kg body weight) to induce diabetes. Non-diabetic rats served as normal controls. The diabetic rats were distributed into 2 experimental groups (3 rats per group). Urine glucose levels were measured weekly with Tes-Tape (Eli Lilly Inc.). One group of rats was treated by oral gavage of Compound 4 (30 mg/kg body weight) daily for 21 days; the Compound 4 was suspended in 2% carboxymethyl cellulose and polytron/homogenized. The normal & diabetic vehicle-treated (control) groups were oral gavaged with vehicle alone daily.

Rat peritoneal macrophages were harvested with 10 ml RPMI 4 days after intraperitoneal injection of 10 ml of thioglycolate (3% wt/vol). Macrophages were then purified by density gradient centrifugation (lymphoprep) and cell numbers were counted in a hemocytometer. The cells were then tested in a cell migration assay (Cell BioLabs, Inc, CytoSelect 96-well with 5 um fluorometric format) using conditioned medium containing inflammatory mediators including MCP-1 from LPS-macrophages as a chemoattractant. The macrophages from different groups of rats were also plated onto 24 well-plates with and without p.g.LPS challenge for 24 hours in 37° C., 5% CO₂. Conditioned medium were harvested for measurement of cytokine levels (IL-6 and IL-13) & MMPs (gelatin zymography).

### Results

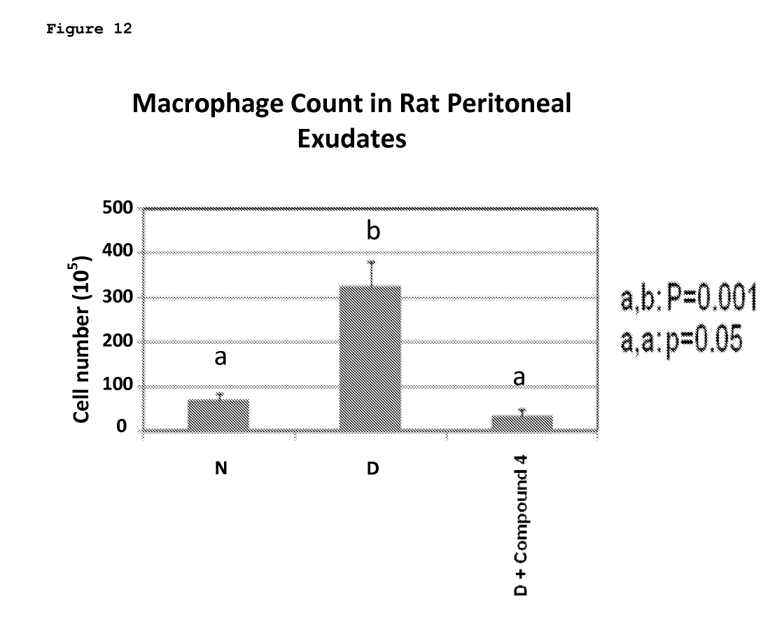

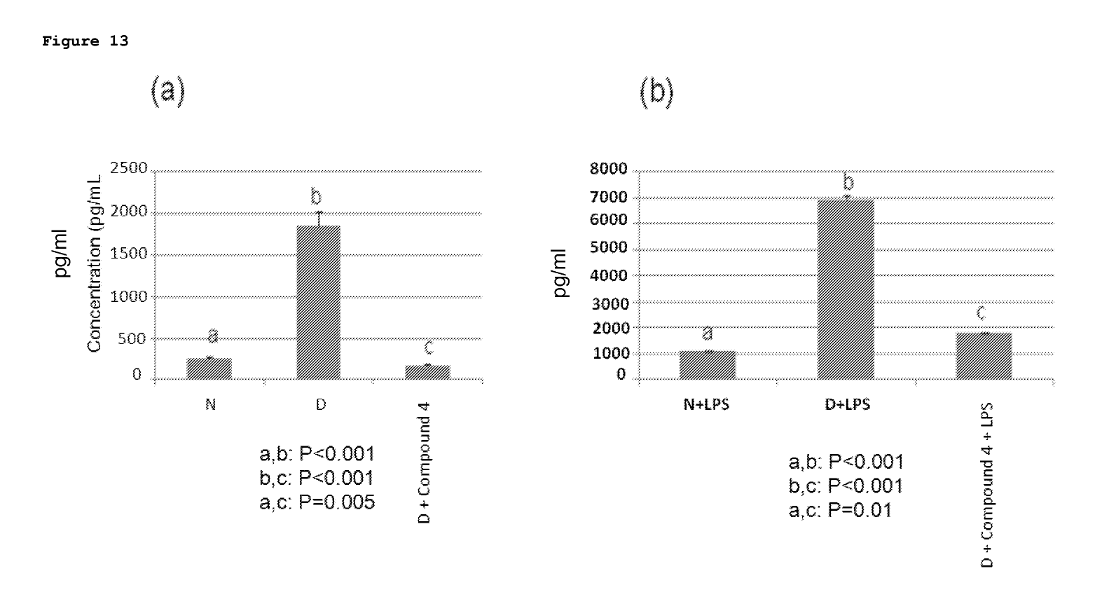

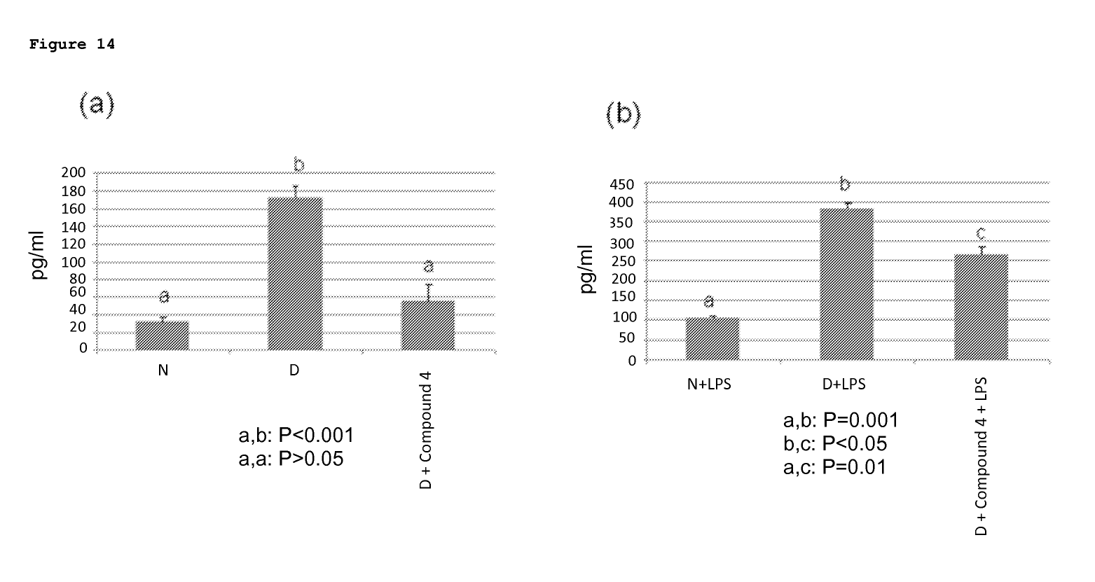

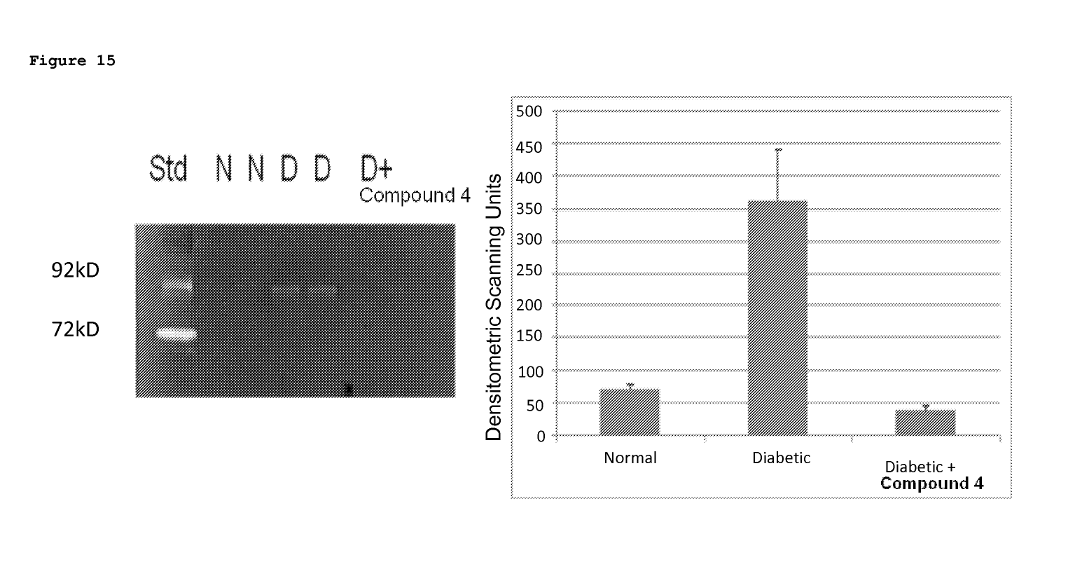

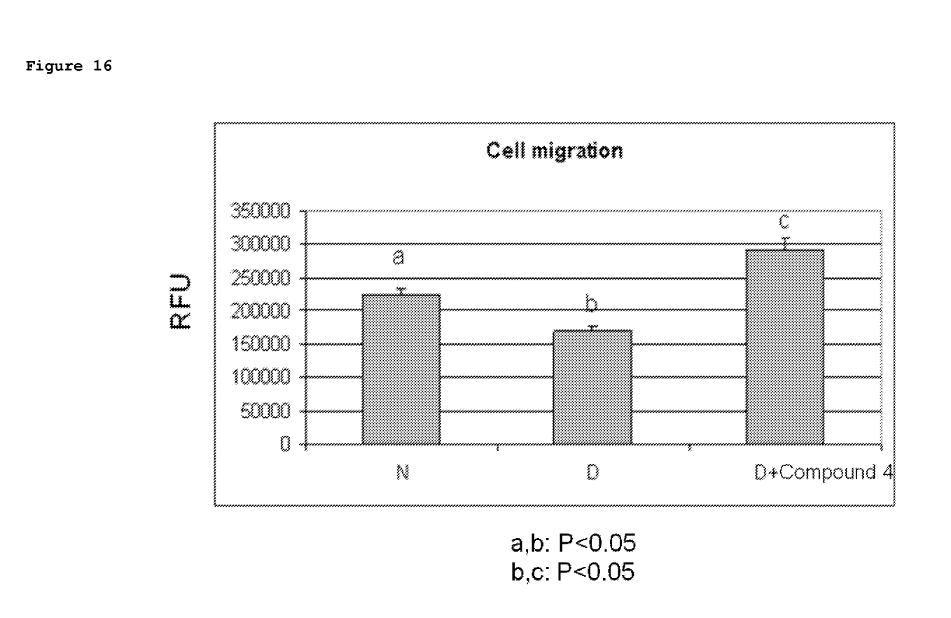

Oral administration of Compound 4 decreased pathologically excessive macrophage accumulation in periotoneal exudates of diabetic rats but did not affect the severity of hyperglycemia in the diabetics (FIG. 12). Additionally, oral administration of Compound 4 decreased excessive inflammatory cytokine (IL-6 IL-1β) production (FIGS. 13 and 14), and MMP-9 levels in the peritoneal macrophages of diabetic rats (FIG. 15). Macrophages in normal rats showed normal chemotactic activity. In diabetic rats, the chemotactic activity of of macrophages was suppressed. Oral administration of Compound 4 counteracted the suppression of macrophage chemotactic activity in diabetic rats (FIG. 16).

### Example 8

### Compound 4 Homologues are Well Tolerated and Effective

The experiments described hereinabove in Examples 2-7 are also performed using Compound 4 homologues. Compound 4 homologues are defined hereinabove at the beginning of the Detailed Description of the Invention. The Compound 4 homologues are highly active against MMPs 1, 2, 3, 7, 8, 9, 12, 13 and 14, yet show virtually insignificant toxic effects in diabetic rats.

### Skin Wound Healing

Both oral and topical administration of a Compound 4 homologue enhances wound healing in normal and diabetic rats.

### Inflammatory Response

Compound 4 homologues attenuate the prolonged inflammatory response in diabetic rats. Oral administration of a Compound 4 homologue decreases pathologically excessive macrophage accumulation in periotoneal exudates of diabetic rats but does not affect the severity of hyperglycemia in the diabetics. Additionally, oral administration of a Compound 4 homologue decreases pathologically excessive IL-6 IL-1β production, and MMP-9 levels in the peritoneal macrophages of diabetic rats. Oral administration of a Compound 4 homologue also counteracted the suppression of macrophage chemotactic activity in diabetic rats.

### Example 9

### Compound 4 and Compound 4 Homologues are Effective to Treat Contact Dermatitis and Insect Bites

The leaves of a poison ivy plant are applied to equally sized areas of the left and right arms of a human subject. After contact dermatitis presents with itching on each arm as a result of contact with the poison ivy, Compound 4 or a Compound 4 homologue is topically administered to the contact dermatitis on one arm, and Compound 2 is applied to the contact dermatitis on the other arm. The contact dermatitis that is treated with Compound 4 or a Compound 4 homologue stops itching sooner and heals quicker than the contact dermatitis that is treated with Compound 2.

Mosquitoes are presented with and allowed to bite the left and right arms of a human subject. After the bites begin to itch, Compound 4 or a Compound 4 homologue is topically administered to the mosquito bites on one arm, and Compound 2 is applied to the bites on the other arm. The mosquito bites that are treated with Compound 4 or a Compound 4 homologue stop itching sooner and heal quicker than the bites treated with Compound 2.

### Example 10

### Effect of Compound 4 on Newly-Synthesized Collagen in Wounds of Diabetic Rats

Biochemical measurements were made on wound tissue 6 days after creating the standardized 6 mm diameter wounds in the various groups of rats including: (1) non-diabetic controls (NDC); (2) Diabetic (DM) rats treated topically with 0.25% curcumin; (3) DM treated with 1% curcumin; (4) DM treated with 0.25% Compound 4, and (5) DM rats treated with 1% Compound 4. The wound tissues from these five different groups were then hydrolyzed (after extraction; see below) to degrade the collagen to individual amino acids, and the amino acid unique to collagen, hydroxyproline, was measured by a commercial colorimetric assay. Prior to hydrolysis, the wound tissues were extracted (at 4° C. to maintain the triple-helical conformation of the collagen molecules) to obtain first the salt-soluble fraction, then the acid-soluble fraction, leaving the insoluble fraction. Based on the collagen literature, the salt-soluble fraction (SSF) represents the newly-synthesized uncrosslinked collagen molecules; the acid-soluble fraction (ASF) is an older fraction which begins to exhibit Schiff-base intramolecular crosslinks; and the insoluble fraction (IF) is the oldest fraction characterized by greater numbers of intramolecular as well as intermolecular covalent crosslinks.

### Results

As shown in Table 2, inducing diabetes decreased the proportion (%) of the collagen in the wound tissue, in the “SSF”, compared to the non-diabetic control rats. This indicated that diabetes decreases the relative amount of newly-synthesized collagen in the wound tissue (consistent with previous publications) compared to the levels of this poorly-crosslinked collagen in the wounds of the NDC/normal rats. Moreover, all of the topical treatments in this experiment, including both curcumin and Compound 4, dramatically increased the proportion of salt-soluble newly-synthesized collagen in the wound tissue of the diabetic rats. However, consistent with previous data on these two compounds, the 0.25% and 1% preparations of Compound 4 were more effective, than the same concentrations of curcumin, in their ability to increase the levels of newly-synthesized collagen in the tissue of this animal model of impaired wound healing.

TABLE 2
The Effect of Topical Compound 4 on Newly-Synthesized
Collagen in Wounds of Severely Diabetic Rats

| | Collagen Fractions (%) | | | Blood |
|---|---|---|---|---|
| | Salt- | | | Glucose |
| Experiment Group | soluble | Acid-soluble | Insoluble | (mg/dL) |
| NDC | 16.3 | 49.5 | 34.2 | 139 |
| Diabetics (DM) | 12.0 | 39.3 | 48.7 | 560 |
| DM + 0.25% curcumin | 15.3 | 48.3 | 36.4 | 569 |
| DM + 1% curcumin | 22.0 | 50.9 | 27.1 | 540 |
| DM + | 26.6 | 41.7 | 31.7 | 538 |
| 0.25% Compound 4 | | | | |
| DM + | 30.1 | 29.4 | 40.5 | 545 |
| 1% Compound 4 | | | | |

### Discussion

### Skin Wounds

In vitro cell and tissue culture data, and in vivo data collected after administering Compound 4 systemically once/day by oral intubation was found to exhibit greater efficacy as a matrix metalloproteinase- and cytokine-modulator than other related zinc-binding inhibitor compounds (Chemically Modified Curcumins and BAMs/bis-aroyl methanes). See, e.g. Table 1. Furthermore, Compound 4 was well tolerated and not toxic compared to related compounds such as Compound 3. The objective of the studies exemplified hereinabove was to administer the “lead” compound, Compound 4, topically and systemically to a rat model of type I diabetes to determine whether these treatments improved impaired wound healing in skin, a well-known complication of diabetes and other inflammatory diseases.

Of significant interest is that the therapeutic benefit of the ability of topical Compound 4 to increase the levels of newly-synthesized collagen in the wounded skin occurred even in diabetic rats which showed extremely high blood glucose levels (i.e., very severe diabetes) throughout the experimental protocol (see, e.g. Table 2).

### Conclusions and Proposed Mechanisms

The clinical, biochemical and histologic data generated in the examples all indicate that Compound 4, particularly when topically applied as a 1% suspension, promotes wound healing, including in diseases such as poorly-controlled diabetes. Without wishing to be bound by any scientific theory, Compound 4 appears to produce this beneficial effect by “resolving” (rather than inhibiting) the inflammatory phase of wound healing which could contribute to an accelerated connective tissue (collagen)-restoration phase. The former may reflect the ability of the compound to “normalize” the suppressed function of macrophages and other inflammatory cells resulting from the hyperglycemic diabetic state. Moreover, the latter may reflect an attenuation of the excessive MMP activity seen in the wounds and produced by macrophages from the peritoneal exudates in the diabetic rats (excessive MMP activity would be expected to preferentially degrade the newly synthesized, poorly cross-linked collagen, rather than the older more polymerized collagen fibrils which would inhibit the repair of the connective tissues of the dermis). Again, without wishing to be bound by any scientific theory, a proposed model illustrates these events in FIG. 11.

### Oral Wounds

The present invention provides novel compounds which can inhibit tissue destructive enzymes (proteinases) and inflammatory mediators (cytokines) involved in inflammatory and other conditions in the oral cavity and skin. However, some of these zinc-binding compounds may be relatively insoluble. The present invention also provides a method to solubilize these compounds with a non-toxic organic solvent, such as N-methyl glucamine, which is related to a non-sugar sweetener (glucitol).

A compound of the invention and N-methyl glucamine can be formulated into a pleasant-tasting non-cariogenic mouthrinse which does not require alcohol (alcohol can increase susceptibility to oral cancer) and which could be an effective treatment for a variety of oral conditions including (but not limited to) periodontitis, root caries, mucositis, and blistering lesions. Other formulations could be useful as a toothpaste orally, or to enhance wound healing or the healing of other conditions in skin.

### REFERENCES

1. Gross J and Lapiere CM. Collagenolytic activity in amphibian tissues: a tissue culture assay. Proc. Natl. Acad. Sci., 48: 1014-1022, 1962.
2. Sorsa T, Tjäderhane L, Konttinen Y T, Lauhrio A, et al. Matrix metalloproteinases: contribution to pathogenesis, diagnosis and treatment for periodontal inflammation. Ann. Med., 38: 306-321, 2006.
3. Roy R, Yang J and Moses M A. Matrix metalloproteinases as novel biomarkers and potential therapeutic targets in human cancer. J. Clin. Oncol., 27: 5287-5297, 2009.
4. Golub et al. (1998) “Tetracyclines Inhibit Connective Tissue Breakdown by Multiple Non-Antimicrobial Mechanisms” Adv. Dent. Res., 12, pp. 12-26.
5. Close, D R. (2001) “Matrix metalloproteinase inhibitors in rheumatic diseases.” Ann. Rhuem. Dis., 60, pp. iii62-iii67.
6. (a) Dove, A. (2002) “MMP inhibitors: Glimmers of hope amidst clinical failures.” Nature Med., 8(2), p. 95. (b) Trivedi N R, Gilliland K L, Zhao W L, et al (2006). Gene Array expansion profiling in acne lesions reveals marked upregulation of genes involved in inflammation and matrix remodeling. Joural of Invest. Derm. 126(5): 1071-1079; (c) Papakonatantinov E, Aletius A J, Glass E, et al. (2005). Matrix Metalloproteinases of epitheilial origin in facial sebum of patients with acne and their regulationby isotretindin. Journal of Invest. Derm 125(4):673-684.
7. Borkakoti, N. (2004) “Matrix metalloprotease inhibitors: design from structure.” Biochem Soc. Trans., vol. 32, part 1, pp. 17-20.
8. Peterson, J T. (2006) “The importance of estimating the therapeutic index in the development of matrix metalloproteinase inhibitors.” Cardio. Res., 69, pp. 677-687.
9. Bachmeier, B E, et al. (2008) “Curcumin downregulates the inflammatory cytokines CXCL1 and -2 in breast cancer cells via NFkB.” Carcinogenesis, 29(4), pp. 779-789.
10. Kaur, G, et al. (2006) “Inhibition of oxidative stress and cytokine activity by curcumin in amelioration of endotoxin-induced experiemental hepatotoxicity.” Clin. Exp. Immunol., 145, pp. 313-321.
11. Begum, A N, et al. (2008) “Curcumin structure-function, bioavailability, and efficacy in models of neuroinflammation and Alzheimer's disease.” J. Pharm. Exp. Ther., 326(1), pp. 196-208.
12. Banerji, A, et al. (2004) “Effect of curcumin on gelatinase A (MMP-2) activity in B16F10 melanoma cells.” Cancer Lett., 211(2), pp. 235-242.
13. Woo, M.-S., et al. (2005) “Curcumin suppresses phorbol ester-induced matric metalloproteinase-9 expression by inhibiting the PKC to MAPK signaling pathways in human astroglioma cells.” Biochem. Biophys. Res. Comm., 335, 1017-1025.
14. Lin, L, et al. (2006) “Antitumor Agents. 250. Design and Synthesis of New Curcumin analogues as Potential Anti-prostate cancer Agents.” J. Med. Chem., 49(13), pp. 3963-3972.
15. a) Lee et al. U.S. Pat. No. 7,355,081; b) Shih et al. PCT International Application Publication WO 2008/085984.
16. Safavy et al. PCT International Application Publication WO 2008/051474.
17. Van Der Jagt et al. US Patent Application Publication 2006/0276536.
18. Pandol et al. U.S. Pat. No. 7,060,733.
19. Baum, L; Ng, A. (2004) “Curcumin interaction with copper and iron suggests one possible mechanism of action in Alzheimer's disease animal models.” Journal of Alzheimer's Disease, 6(4), pp. 367-377.
20. Heng, M. PCT International Application Publication WO 99/42094.
21. Singer, A. et al. (2007) “Curcumin Reduces Burn Progression in Rats,” Acad. Emerg. Med., 14, pp. 1125-1129.
22. Sidhu, G S. et al. (1998) “Enhancement of wound healing by curcumin in animals,” Wound Rep. Reg., 6, pp. 167-177.
23. Sidhu, G S. et al. (1999) “Curcumin enhances wound healing in streptozoticin induced diabetic rats and genetically diabetic mice,” Wound Rep. Reg., 7, pp. 362-374.
24. Phan, T.-T. et al. (2001) “Protective effects of curcumin against oxidative damage on skin cells in vitro: Its implication for wound healing,” J. Trauma, 51, pp. 927-931.
25. Budnikova, M V; Rubinov, D B. (2001) “Synthesis of Endocyclic Enol Methyl Ethers of 3-Acylthiotetronic Acids and Their Reactions with Amines,” Russian Journal of Organic Chemistry, 37(10), pp. 1478-1485.
26. Saito, K; Sato, T. (1978) “Diversity in the base-induced photoreactions of 3-acetyl-2,4-dioxothiolane (3-acetylthiotetronic acid).” Chem. Lett., pp. 307-310.
27. Sorsa T and Golub L M. (2005) “Is the excessive inhibition of matrix metalloproteinases (MMPs) by potent synthetic MMP inhibitors (MMPIs) desirable in periodontitis and other inflammatory diseases? That is: ‘Leaky’ MMPIs vs excessively efficient drugs” Oral Dis., 11, pp. 408-409.
28. Golub et al. (1996) “New therapeutic uses for an old family of drugs: travels of a dental researcher from the lab to the university's office of technology transfer and beyond.” Technol. Health Care, 4, pp. 311-316.
29. Liu et al. (2001) “The Lipophilicity, Pharmacokinetics, and Cellular Uptake of Different Chemically-Modified Tetracyclines (CMTs).” Curr. Med. Chem., 8, pp. 243-252.
30. Yu Z et al. (1993) “Chemically-modified tetracycline normalizes collagen metabolism in diabetic rats: a dose-response study.” J Periodontal Res., 28(6 Pt 1), pp. 420-428.
31. Ryan M E et al. (2001) “Excessive matrix metalloproteinase activity in diabetes: inhibition by tetracycline analogues with zinc reactivity.” Curr Med Chem., 8(3), pp. 305-316.
32. Ryan M E et al. (1999) “MMP-mediated events in diabetes.” Ann N Y Acad Sci., 878, pp. 311-334.
33. Ramamurthy N S, Zebrowski E J & Golub L M. (1973) “Collagenolytic activity of alloxan diabetic rat gingivae.” Diabetes, 22, pp. 272-274.
34. Golub L M, Greenwald R A, Zebrowski E J & Ramamurthy N S. (1978) “The effect of experimental diabetes on the molecular characteristics of rat tail tendon collagen.” Biochim. Biophys. Acta, 534, pp. 73-81.
35. Schneir, M. and Golub, L. M. (1981) “The effect of streptozotocin diabetes on collagen metabolism.” In Agarwal, M. K. (Ed.): Streptozotocin: Fundamentals and Therapy. Elsevier/North Holland Biomedical Press, pp. 161-182.
36. Schneir M, Ramamurthy N S & Golub L M. (1985) “Dietary ascorbic acid normalized diabetes-induced underhydroxylation of nascent type I collagen molecules.” Collagen Rel. Res., 5, pp. 415-422.
37. Leung M K, Folkes G A, Ramamurthy N S & Golub, L M. (1986) “Diabetes stimulates procollagen degradation in rat tendon in vitro.” Biochim. Biophys. Acta, 880, pp. 147-152.
38. Schneir M, Imberman M, Ramamurthy NS & Golub LM. (1988) “Streptozotocin-induced diabetes and the rat periodontium: Decreased relative collagen production.” Collagen Rel. Res., 8, pp. 221-232.
39. Schneir M, Ramamurthy N S & Golub L M. (1990) “Minocycline treatment of diabetic rats increases skin collagen production and mass.” Matrix, 10, pp. 112-123.
40. Golub L M, Ramamurthy N S, Lee H M & Rifkin, B. (1990) Tetracycline administration prevents diabetes induced osteopenia in the rat.” Res. Commun. Chem. Path. Pharmacol., 68, pp. 27-40.
41. Kaneko H, Sasaki T, Ramamurthy N S & Golub L M. (1990) “Tetracycline administration normalizes the structure and acid phosphatase activity of osteoclasts in streptozotocin-diabetic rats.” Anatom. Rec., 227, pp. 427-436.
42. Golub L M, Evans R T, McNamara T F et al. A non-antimicrobial tetracycline inhibits gingival metalloproteinases and bone loss in Porphyromonas gingivalis-induced periodontitis in rats. Ann. N.Y. Acad. Sci., 732: 96-111, 1994.
43. Blankenberg, S, Rupprecht H J, Poirier O, Bickel C, Smieja M, Hafner G, Meyer J, Cambren F, Tiret L and AtheroGene Investigators. Plasma concentrations and genetic variation of matrix metalloproteinase-9 and prognosis of patients with cardiovascular disease. Circulation 107: 1579-1585, 2003.
44. Patel R V, Clark L N, Lebwohl M, Weinberg J M. Treatment for psoriasis and the risk of malignancy. J Am Acad Dermatol 60(6):1001-17, 2009.
45. Bingham, S. J., Tyman, J. H. P. Improved Synthesis of Alkyl Diacetylacetates. Organic Preparations and Procedures Int. 33, 357-361 (2001).
46. Dieckmann, W., Hoppe, J., Stein, R. On the Behavior of 1,3-Dicarbonyl Compounds with Phenyl Isocyanate. Chem. Ber. 37, 4627-4638 (1904).
47. Pabon H. H. J. Synthesis of Curcumin and Related compounds. Rec. Tray. Chim. 83, 379-386, (1964).
48. Ruby A J, Kuttan G, Badu K D, Rajasekharan K N, Kuttan R. Anti-tumour and antioxidant activity of natural curcuminoids. Cancer Lett. 94:79-83 (1995).
49. Golub L, Lee H M, Lehrer G, et al., Minocycline reduces gingival collagenolytic activity during diabetes. J. Periodontal Res. 18:516-526 (1983)
50. Golub L M, Ciancio S G, Ramamurthy N S, et al., Low-dose Doxycycline Therapy: Effect on Gingival and Crevicular Fluid Collagenase Activity in Humans. J. Periodont Res. 25:321-330 (1990).
51. Lee H M, Ciancio S G, Tuter G et al., Subantimicrobial dose doxycycline efficacy as a matrix metalloproteinase inhibitor in chronic periodontitis is enhanced when combined with a non-steroid anti-inflammatory drug. J. Periodontol. 75:453-463 (2004)

1. A compound having the structure 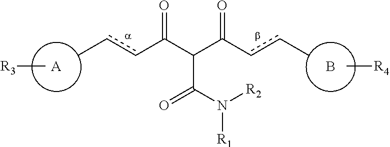
wherein
bond α and β are each, independently, present or absent;
A is an aromatic or heteroaromatic ring; B is an aromatic or heteroaromatic ring;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or 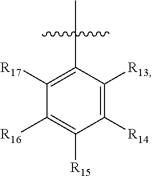
wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S)R₁₈, —C(═NH)R₁₈, C(═NR₁₈)R₁₈, —C(═N)R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
wherein Z is halogen, and
R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
wherein R₂₁ is, H, —CF₃, —CH₃, C₁₋₅ alkyl, C₂₋₅ alkenyl, C₂₋₅ alkynyl, or —C(═O)-heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂)(OR₂₃), or —P(OR₂₂) (OR₂₃),
wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted; and
when α and β are present, A is phenyl with R₃ at the para position, B is phenyl with R₄ at the para position, R₂ is H, R₃ is —OH or —N(CH₃)₂, and R₄ is —OH or —N(CH₃)₂, then R₁ is other than unsubstituted phenyl,
or a salt thereof.

2. The compound of claim 1 having the structure 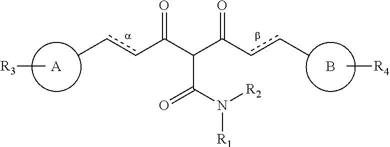
wherein
bond α and β are each, independently, present or absent;
X is —CH, —CR₃ or N; Y is —CH, —CR₄ or N;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or 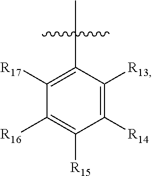
wherein R₁₃, R₁₄, R₁₅, R₁₆, and R₁₇ are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S)R₁₈, —C(═NH)R₁₈, C(═NR₁₈)R₁₈, —C(═N)R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
wherein Z is halogen, and
R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H, —CH₃, —OR₂₁, —CO₂R₂₁, —CF₃, C₁₋₅ alkyl, C₂₋₅ alkenyl, C₂₋₅ alkynyl, aryl, heteroaryl, or heterocyclyl,
wherein R₂₁ is, H, —CF₃, —CH₃, C₁₋₅ alkyl, C₂₋₅ alkenyl, C₂₋₅ alkynyl, or —C(═O)-heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂)(OR₂₃), or —P(OR₂₂) (OR₂₃),
wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted; and
when α and β are present, X is —CH, Y is —CH, R₂ is H, R₃ is —OH or —N(CH₃)₂ at the para position, and R₄ is —OH or —N(CH₃)₂ at the para position, then R₁ is other than unsubstituted phenyl,
or a salt thereof.

3. The compound of claim 2, wherein X is N, or a salt thereof.

4. The compound of claim 2, wherein α and ρ are both present, or a salt thereof.

5. The compound of claim 2 having the structure 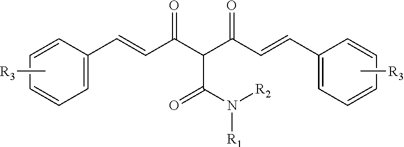
wherein
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or 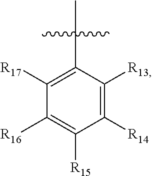
wherein R₁₃, R₁₄, R₁₅, R₁₆ and R₁₇ and are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S)R₁₈, —C(═NH)R₁₈, C(═NR₁₈)R₁₈, —C(═N)R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
wherein Z is halogen, and
R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H or —CH₃; and
R₃ and R₄ are each —OR₂₂,
wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)— heterocyclyl, and
wherein each occurrence in the compound of alkyl, alkenyl, or alkynyl is branched or unbranched, unsubstituted or substituted,
or a salt thereof.

6. The compound of claim 5 having the structure 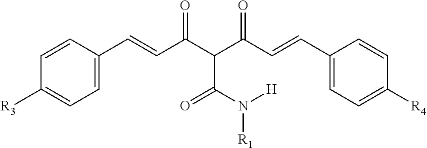
wherein
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, heterocyclyl, or 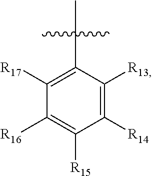
wherein R₁₃, R₁₄, R₁₅, R₁₆ and R₁₇ and are each independently, H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S)R₁₈, —C(═NH)R₁₈, C(═NR₁₈)R₁₈, —C(═N)R₁₈, —POR₁₈, —P(═O)(OR₁₈)(OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
wherein Z is halogen, and
R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each —OR₂₂,
wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)— heterocyclyl, and
wherein each occurrence in the compound of alkyl, alkenyl, or alkynyl is branched or unbranched, unsubstituted or substituted,
or a salt thereof.

7. The compound of claim 5, wherein R₂₂ is H, C₁₋₅ alkyl, C₂₋₅ alkenyl, C₂₋₅ alkynyl, aryl, heteroaryl, heterocyclyl, or —C(═O)-heterocyclyl, wherein each occurrence of alkyl, alkenyl, or alkynyl is branched or unbranched, unsubstituted or substituted, or a salt thereof.

8. The compound of claim 2 having the structure 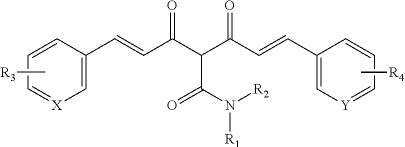
wherein
X is —CH, —CR₃ or N; Y is —CH, —CR₄ or N;
R₁ is pyridine, imidazol, oxazole, thiazole, pyridine, pyridazine, pyrazine, pyrimidine, morpholine, C₁₋₁₀ alkyl, C₂₋₁₀ alkenyl, C₂₋₁₀ alkynyl, aryl, heteroaryl, or heterocyclyl;
R₂ is H or —CH₃; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂)(OR₂₃), or —P(OR₂₂) (OR₂₃),
wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

9. The compound of claim 2 having the structure 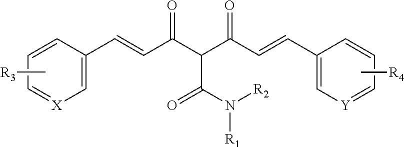
wherein
X is —CH, —CR₃ or N; Y is —CH, —CR₄ or N;
R₁ is 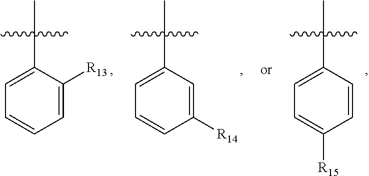
wherein R₁₃, R₁₄, or R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S) R₁₈, —C(═NH) R₁₈, C(═NRs) R₁₈, —C(═N)R₁₈, —POR₁₈, —P(═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
wherein Z is halogen, and
R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl;
R₂ is H or —CH₃; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

10. The compound of claim 9 having the structure 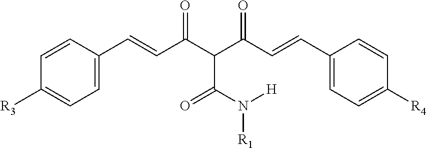
wherein
R₁ is 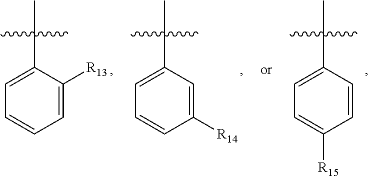
wherein R₁₃, R₁₄, or R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S)R₁₈, —C(═NH) R₁₈, C(═NR₁₈) R₁₈, —C(═N) R₁₈, —POR₁₈, —P(═O) (OR₁₈) (OR₁₉), —P(OR₁₈) (OR₁₉), —C(═S)R, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
wherein Z is halogen, and
R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each independently, halogen, —NO₂, —NR₂₂R₂₃, —NHR₂₂R₂₃⁺, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂) (OR₂₃),
wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

11. The compound of claim 10, wherein
R₁ is 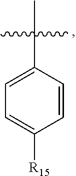
wherein R₁₅ is H, halogen, —OCZ₃, —CZ₃, —OCH₂CH(OH)CH₂OH, —OCH(CH₂OH)₂, —NO₂, OCH₃, —CN, —NR₁₈R₁₉, —SR₁₈, —CO₂R₁₈, —OR₂₀, —COR₂₀, —CSR₂₀, —NHCOR₁₈, —SOR₁₈, —POR₁₇, —C(═S)R₁₈, —C(═NH)R₁₈, C(═NR₁₈)R₁₈, —C(═N)R₁₈, —POR₁₈, —P((═O) (OR₁₈) (OR₁₉), —P(OR₁₈) (OR₁₉), —C(═S)R₁₈, —NHR₁₈R₁₉⁺, —C(═O)-heterocyclyl, C₁₋₁₀ alkyl, C₂₋₁₀ alkenyl, C₂₋₁₀ alkynyl, aryl, heteroaryl, or heterocyclyl,
wherein Z is halogen, and
R₁₈, R₁₉, and R₂₀ are each, independently, H, C₁₋₁₀ alkyl, C₂₋₁₀ alkenyl, C₂₋₁₀ alkynyl, aryl, heteroaryl, or heterocyclyl; and
R₃ and R₄ are each independently, halogen, —NO₂, —NR₂₂R₂₃, —NHR₂₂R_{23′}, —SR₂₂, —SO₂R₂₂, —SO₃R₂₂, —OR₂₂, —CO₂R₂₂, —CF₃, —POR₂₂, —P(═O) (OR₂₂) (OR₂₃), or —P(OR₂₂)(OR₂₃),
wherein R₂₂ and R₂₃ are each, H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

12. The compound of claim 11, wherein R₃ and R₄ are each —OR₂₂, wherein R₂₂ is H, —CF₃, —CH₃, C₁₋₅ alkyl, C₂₋₅ alkenyl, C₂₋₅ alkynyl, aryl, heteroaryl, heterocyclyl, or —C(═O)-heterocyclyl, and
wherein each occurrence in the compound of alkyl, alkenyl, or alkynyl is branched or unbranched, unsubstituted or substituted,
or a salt thereof.

13. The compound of claim 12, wherein R₂₂ is H.

14. The compound of claim 13, having the structure 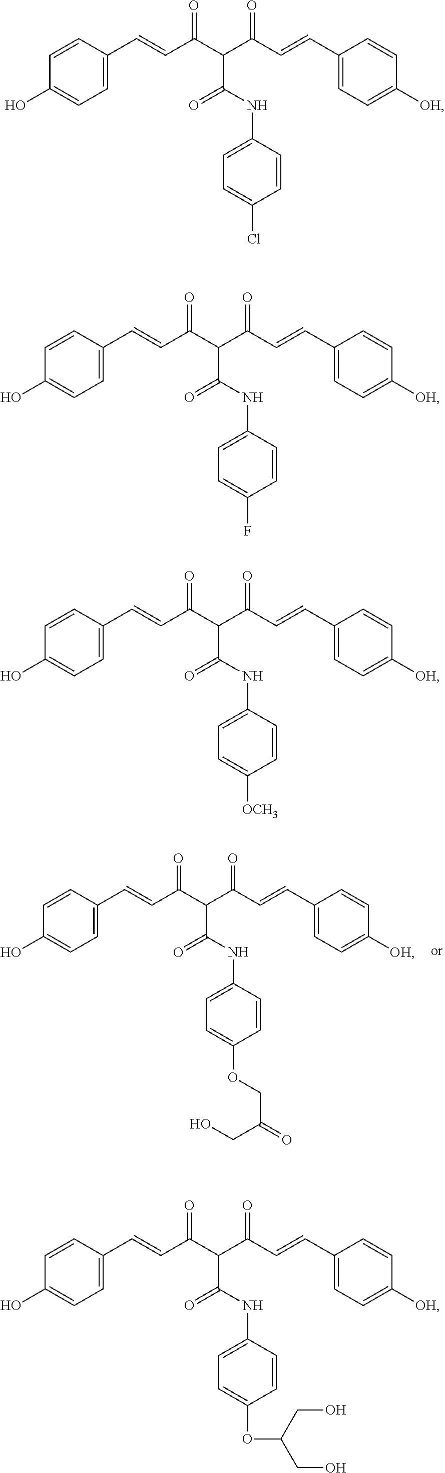
or a salt thereof.

15. A pharmaceutical composition comprising a pharmaceutically acceptable carrier and the compound of claim 1.

16-54. (canceled)
